(19) United States
(12) Patent Application Publication
(10) Pub. No.: US 2011/0190307 A1
(43) Pub. Date: Aug. 4, 2011

(54) Assay

(76) Inventors: Catherina G. Becker, Edinburgh (GB); Thomas Becker, Edinburgh (GB)

(21) Appl. No.: 13/001,229

(22) PCT Filed: Jun. 25, 2009
(86) PCT No.: PCT/GB2009/001589

§ 371 Date: Apr. 18, 2011

(30) Foreign Application Priority Data
Jun. 25, 2008 (GB) 081642.8

(51) Int. Cl. A61K 31/519; C12Q 1/02; C07D 221/18; C07D 211/88; C07D 305/14; C07C 57/26; C07D 475/08; C07D 309/30; C07D 311/78; C07D 335/04; A61K 31/473; A61K 31/45; A61K 31/337; A61K 31/203; A61K 31/366; A61K 31/352; A61K 31/382; A61P 25/00; A61P 25/28

(52) U.S. Cl. 514/249; 435/29; 546/75; 546/220; 549/510; 562/510; 544/260; 549/292; 549/384; 549/24; 514/284; 514/328; 514/449; 514/559; 514/460; 514/453; 514/432

(57) ABSTRACT

The present invention provides zebrafish based methods for the identification of compounds potentially useful in the treatment of motor neuron degenerative diseases (MNDDs), compounds identified by these methods and compositions, methods and medicaments for treating MNDDs.

### FIELD OF THE INVENTION

The present invention provides methods for the identification of compounds potentially useful in the treatment of motor neuron degenerative diseases (MNDD), compounds identified thereby and methods, compositions and medicaments for treating the same.

### BACKGROUND OF THE INVENTION

Damage to the spinal cord by injury or motor neuron diseases is devastating because lost neurons are not replaced in the adult mammalian spinal cord (Bareyre, 2007; Ninkovic and Gotz, 2007). Adult zebrafish have an impressively high regenerative capacity. This includes heart tissue regeneration (Poss et al., 2002), retinal regeneration (Bernardos et al., 2007; Fimbel et al., 2007) and functional spinal cord regeneration (Becker et al., 2004).

There is significant neurogenesis in specific neurogenic zones even in the unlesioned brain of adult zebrafish (Zupanc et al., 2005; Adolf et al., 2006; Chapouton et al., 2006; Grandel et al., 2006). This is similar to mammals, which probably have fewer of these zones (Gould, 2007). However, the unlesioned adult zebrafish spinal cord shows very little, if any, proliferation and neurogenesis (Zupanc et al., 2005; Park et al., 2007). Therefore, a prerequisite for motor neuron regeneration would be plasticity of relatively quiescent spinal progenitor cells after injury.

Following these observations lesion-induced neuronal regeneration in the heavily myelinated spinal cord of the fully adult zebrafish (>4 months) after complete spinal transection prompted was investigated.

In the UK, the incidence of amyotrophic lateral sclerosis (ALS) is 2 in 1000,000 and patients diagnosed with ALS have a life expectancy of 2-3 years. Riluzole is the only drug available to treat this disease but it merely slows disease progression rather than stopping it.

As such there is a need for new therapeutic agents capable of treating diseases which involve loss of, damage to or the degeneration of motor neurons through disease or injury.

### SUMMARY OF THE INVENTION

The present invention is based on the observation that adult zebrafish modified to include a spinal cord lesion are capable of regenerating motor neurons. This is in contrast to the mammalian spinal cord which cannot regenerate motor neurons lost through injury or disease. In view of this remarkable observation, the present inventors are exploiting zebrafish with spinal lesions to identify agents potentially useful in the treatment of motor neuron degenerative diseases (MNDD).

Accordingly, and in a first aspect, the present invention provides a method of identifying compounds potentially useful in the treatment of motor neuron degenerative diseases (MNDD), said method comprising the step of contacting a test agent with a zebrafish having a spinal lesion and determining the effect of said test agent on the growth, differentiation, development and/or regeneration of motor neurons.

The method provided by this first aspect will be referred to hereinafter as the “validation screen”

The present inventors have determined that test agents potentially useful in the treatment of MNDDs are capable of modulating the motor neurone regeneration which occurs following the introduction of a spinal lesion in a zebrafish. In particular, potentially useful test agents modulate the growth, differentiation, development and/or regeneration of motor neurons in the vicinity of the spinal lesion and facilitate rapid restoration of spinal cord function.

The term “modulate” should be understood as encompassing an increase and/or decrease in the level of motor neuron growth, differentiation, development and/or regeneration. Preferably, agents which may have therapeutic benefit in the treatment of MNDDs, bring about an increase in the level of motor neuron, differentiation, development and/or regeneration which occurs following the introduction of a spinal lesion.

Modulation of motor neuron growth, differentiation, development and/or regeneration in the validation screen may manifest in the form of newly generated motor-neurons appearing in the vicinity of the spinal lesion, the appearance of newly generated BrdU+ motor neurons and/or fully differentiated ChaT+ motor neurons having synapse-like contacts or processes. In addition, the modulation of motor neuron growth, development and/or differentiation may also result in the integration of newly formed motor neurons with the existing spinal circuitry. Using the techniques described herein, one of skill could readily detect, monitor and quantify these various processes.

In addition, one of skill will appreciate that by comparing the results obtained from a validation screen conducted in accordance with the methods described herein, with those of a control assay in which a zebrafish modified to include a spinal lesion is either not contacted with a test agent or is contacted with a test agent known to modulate the growth, differentiation, development and/or regeneration of motor neurons, it would be possible to determine and/or quantify the modulatory effect of any given test agent.

Typically, the zebrafish is an adult zebrafish and the spinal lesion comprises a lesion between the vertebrae thereof. In certain embodiments, the lesion may be introduced under anaesthetic and via a longitudinal incision made at the side of the fish to expose the vertebral column. An exemplary method of introducing a spinal lesion into an adult zebrafish is provided in Becker et al., 1997 (‘Axonal regrowth after spinal cord transection in adult zebrafish’; J Comp Neuroscience 377:577-595).

In order to permit the user to detect, visualise, monitor, quantify and/or determine the level of motor axon development, differentiation and/or growth modulation brought about by the test agent or exhibited by the control assay, the zebrafish may be modified in some way so as to render the motor axons/neurons (particularly newly developing motor axons/neurons) labelled or detectable by some means. In one embodiment, the zebrafish used in the validation screen is modified so as to include a gene capable of reporting or feeding back to the user a level of expression of a particular gene. Zebrafish modified in this way may be known as transgenic zebrafish. One of skill will appreciate that genes known to be expressed in motor axons/neurones—particularly developing or actively growing/differentiating motor neurons/axons, may be particularly useful. Such genes may be fused, conjugated or otherwise linked to a reporter gene. Exemplary transgenic zebrafish for use in this aspect of the invention are described below. Additionally, or alternatively, techniques involving the use of compounds capable of binding molecules specific to motor neurons/axons may also be exploited as a means of detecting/labelling these cells. Further details of these techniques are provided below.

The modulatory effects of the test agents on the growth, differentiation, development and/or regeneration of a motor neuron/axon may easily be visualised, determined and/or monitored by any means capable of permitting the identification or visualisation of the label and/or detection means described above. Suitable means may include the use of microscopy such as, fluorescence microscopy

In one embodiment, the test agent is contacted with a zebrafish at one or more time points post spinal lesion formation and in preferred embodiments, the test agents are added at 3, 6 and/or 9 days post lesion formation. One of skill will appreciate that the precise times may vary depending on the nature of the test agent. In addition, while it is possible and perhaps useful to add more than one compound to any given zebrafish, in other embodiments, a single test agent is added to a single zebrafish.

Typically, the modulatory affect of the test agents on the growth, development, differentiation and/or regeneration of motor neurons may be assessed at between 11 and 19 days post lesion, preferably 12-18, more preferably 12-17 and even more preferably at 13-15 days post-lesion. In a preferred embodiment, the modulatory affect of the test agents on the growth, development, differentiation and/or regeneration of motor neurons may be assessed at about 14 days post lesion.

In a further embodiment, the validation screen provided by the first aspect of this invention, may comprise an additional step in which one or more test agents are screened in an embryonic zebrafish, prior to conducting the validation screen described above.

Advantageously, the present inventors have discovered that by first contacting one or more test agents with embryonic zebrafish, and determining the effect of these agents on motor axon development, growth and/or proliferation, it may be possible to rapidly screen a large number of compounds so as to more readily identify those potentially useful in the treatment of MNDDs. Furthermore, this optional first step has the advantage of providing a rapid means of identifying those compounds which are likely not to be useful in the treatment of MNDDs either because they fail to have any modulatory effect on motor neuron growth, differentiation, development and/or regeneration or because they are toxic. In this way less compounds will need to be subjected to the validation screen.

This optional first step will be referred to hereinafter as a “primary screen” which, as stated, may permit the rapid screening of large numbers of any of those test agents described above.

The present inventors have determined that when subjected to the primary screen, those test agents potentially useful in the treatment of MNDDs are capable of modulating the growth, production and/or development of motor axons. Test agents shown to exhibit any of these modulatory effects may be considered as suitable test agents for use in the validation screen. The term “modulate” should be taken to encompass compounds which either promote (i.e. increase) and/or decrease the growth, differentiation and/or development of motor axons. Furthermore, increases and/or decreases in the growth, differentiation and/or development of motor axons may manifest as missing, stunted, excessively branched and/or supernumerary motor axons.

One of skill in the art will appreciate that by comparing the results obtained from a primary screen conducted in accordance with the methods described herein, with those of a control assay in which an embryonic zebrafish is either not contacted with a test agent or is contacted with a test agent known to modulate the growth, differentiation, development and/or regeneration of motor neurons, it may be possible to determine and/or quantify the modulatory effect of any given test agent.

Advantageously, the primary screen may comprise the step of contacting one or more test agents with embryonic zebrafish which have been modified in some way so as to label or render the motor axons/neurons (particularly newly developing motor axons/neurons) detectable by some means. One of skill will appreciate that there are many ways of achieving this and exemplary methods, such as the use of immunological techniques, compounds capable of binding markers specific to motor axons/neurons and transgenic zebrafish are described in detail below.

In order to detect, monitor and/or visualise the modulatory effect of the test agents subjected to the primary screen, one of skill will appreciate that any method capable of detecting the means used to label or detect the motor axons/neurons, may be suitable. By way of example, techniques such as microscopy for example, fluorescence microscopy may be particularly useful.

In one embodiment, the test agents are contacted with the embryonic zebrafish to be used in the primary screen at approximately 3-9 hours post fertilisation (hpf), preferably 4-8 hpf and more preferably 5-7 hpf. In a preferred embodiment, the test agents are contacted with the zebrafish embryos at 6 hpf. It should be understood that while it is possible and perhaps useful to add more than one compound to any given embryonic zebrafish, the primary screening method described here generally requires that a single compound be added to a single zebrafish.

In one embodiment, while the test agents may be added to the zebrafish at between 3-9 hpf (preferably 6 hpf), the effects of said test agents are not determined until approximately 21-27 hpf, preferably 22-26 hpf, more preferably 23-25 hpf. Preferably, the effects of the test agent(s) on the modulation of motor axon development, growth and/or differentiation are determined at 24 hpf.

In addition to the above, the methods provided by this invention may comprise an additional screening step in which, prior to being subjected to the method provided by the first aspect of this invention, and optionally after the primary screen, test agents are contacted with later-stage embryonic zebrafish. This optional step will be referred to hereinafter as a “secondary screen” and is preferably conducted after the primary screen and before the method provided by the first aspect of this invention.

Advantageously, compounds identified via the primary screen as being potentially useful in the treatment of MNDDs may be subjected to the secondary screen so as to further determine which of the potentially useful compounds identified in the primary screen have the greatest therapeutic potential. As will become apparent, the features of the secondary screen are such that those compounds identified as potentially useful in the treatment of MNDDs, more specifically affect motor axon development, growth and/or differentiation and have a greater therapeutic potential. By exploiting the secondary screen, the number of test agents subjected to the validation screen can be reduced.

The inventors have determined that compounds identified via the secondary screen as being of potential therapeutic benefit, modulate the growth, differentiation, development and/or regeneration of motor neurons/axons. Typically, in the secondary screen, the modulation of motor axon/neuron growth, differentiation, development and/or regeneration may manifest as a retardation (i.e. inhibition) or acceleration (i.e. increase) in motor neuron differentiation.

One of skill in the art will appreciate that by comparing the results obtained from a secondary screen conducted in accordance with the methods described herein, with those of a control assay in which an embryonic zebrafish is either not contacted with a test agent or is contacted with a test agent known to modulate the growth, differentiation, development and/or regeneration of motor neurons, it may be possible to determine and/or quantify the modulatory effect of any given test agent.

As with the primary and validation screens detailed above, in order to detect, monitor and/or visualise the motor neuron/axon modulatory effect of any test agents subjected to the secondary screen, it may be desirable to use zebrafish which have been modified in some way so as to render their motor axons/neurons (especially those motor axons/neurons which are actively growing, differentiating and/or regenerating) detectable or labelled by some means. For example, it may be desirable to use the transgenic zebrafish and/or techniques involving the use of compounds capable of binding markers specific to motor axons/neurons (for example immunological techniques) described below.

In order to detect, monitor and/or visualise the modulatory effect of the test agents subjected to the secondary screen, one of skill will appreciate that any method capable of detecting the means used to label or detect the motor axons/neurons, may be suitable. By way of example, techniques such as microscopy for example, fluorescence microscopy may be particularly useful.

In one embodiment, the secondary screen uses embryonic zebrafish at about 21-27 hpf, preferably 22-26 hpf and more preferably 23-25 hpf. In a preferred embodiment, the zebrafish embryos are at 24 hpf. Additionally, or alternatively, the test agents should be added to the zebrafish embryos after completion of early embryogenesis but before the formation of islet-1⁺ motor neurons.

While the test agents may be added to the zebrafish at between 21-27 hpf (preferably 24 hpf), the effects of said test agents are not determined until approximately 45-51 hpf, preferably 46-50 hpf, more preferably 47-49 hpf. Preferably, the effects of the test agent(s) on the modulation of motor axon development, growth and/or differentiation are determined at 48 hpf. Additionally, the effects of the test agent(s) on the modulation of motor axon development, growth and/or differentiation may also be determined at 69-75 hpf, preferably 68-74 hpf, and more preferably at 67-73 hpf. Preferably, the effects of the test agent(s) on the modulation of motor axon development, growth and/or differentiation may also be determined at about 72 hpf. Thus in one embodiment, the effects the effects of the test agent(s) on the modulation of motor axon development, growth and/or differentiation are determined at about 45-51 hpf and at about 69-75 hpf.

Each of the above described methods (the primary screen, the secondary screen and the validation screen) mention the use of techniques which render the motor axons/neurons of zebrafish (both adult and embryonic) and in particular those motor neurons/axons which are actively growing, developing, differentiating and/or regenerating, detectable and/or labelled. In this regard, the zebrafish used in each of the methods described herein may be modified such that the motor axons/motor neurons, particularly developing and/or actively growing/differentiating motor axons/motor neurons, are labelled. For example, the zebrafish may be transgenically modified to include some form of reporter gene construct under the control of genetic elements expressed in motor axons/motor neurons.

Typically, the reporter gene element is capable of reporting a level of activity and/or expression of a particular gene or genes. Suitable reporters elements will be known to one of skill in this field and may include, for example, those which feedback levels of expression/activity via a chemilumiescent or fluorescent reaction or product. By way of example, the gene under the control of genetic elements expressed in motor axons may be fused to a luciferase gene complex, GFP or a membrane-associated (farnesylated) derivative of GFP (mGFP).

Where the zebrafish is to be used in the primary screen, genes suitable for use as reporter gene constructs may include those which provide markers of early motor neuron generation. The secondary screen and/or validation screen may exploit the same genes and/or genes providing markers of more differentiated motor neurons.

In a particularly preferred embodiment, the primary screen may comprise the step of contacting one or more compounds with embryonic zebrafish modified to include a HB9 gene fused to any one of the abovementioned reporter genes. Preferably, the primary screen involves the use of zebrafish modified to include HB9:GFP constructs. One of skill will appreciate that since the HB9 gene is a marker for early motor neuron generation, it is particularly suited for use as gene capable of reporting a the level of early motor neuron development, growth and/or differentiation.

Where the zebrafish are to be used in the secondary screen, they may be modified to include islet-1:GFP reporter constructs. Since Islet-1 provides a marker of late motor neuron development/differentiation, it is particularly suited to this screen.

Where the zebrafish are to be used in the validation step, they may be modified to include HB9:GFP, islet-1:GFP and/or olig2:GFP reporter gene constructs.

Methods of preparing the various transgenic fish potentially useful in this invention are provided by Flanagan-Street et al., 2005 (neuromuscular synapses can form in vivo by incorporation of initially aneural postsynaptic specializations. Development 132:4471-4481: HB9:GFP), Higashijima et al., 2000 (Visualisation of cranial motor neurons in live transgenic zebrafish expressing GFP under the control of the islet-1 promoter enhancer. J neurosci 20:206-218: islet-1:GFP) and Shin et al., 2003 (neural cell fate analysis in zebrafish using olig2 BAC transgenics. Methods cell sci 25:7-14: olig2:GFP).

One of skill in the art will understand that while this invention specifies particular types of transgenic fish that are useful, the invention should in no way be considered as limited to these. One of skill in this field could readily identify other genes suitable for use as reporter genes and prepare and test appropriately modified transgenic fish. Furthermore, each step may involve contacting test agents with one or more of the transgenic fish detailed herein and determining the effects of the test agent(s) on the modulation of motor axon development, regeneration, growth and/or differentiation in each.

In addition, as well as using transgenic fish, it should be understood that other techniques involving the use of compounds cable of binding markers specific for motor neurons/axons may be exploited as a means of detecting/labelling these cell types. Accordingly, any of the methods described herein may use, for example, immunological methods or the like to detect motor neurons and in particular actively growing, developing, regenerating and/or differentiating motor neurons. By way of example, antibodies or other compound which bind to cellular markers specific to motor neurons or to actively growing, developing, regenerating and/or differentiating motor neurons may be exploited as detection means. In one embodiment, antibodies or compounds useful in the detection and/or labelling of motor neurons/axons may include those capable of binding the markers BrdU, ChAT and/or the synaptic marker SV2.

One of skill in the art will appreciate that when using compounds capable of binding cellular markers, such as for example, antibodies, it may be desirable to conduct the screening methods as described above and then to subject the fish to a protocol involving steps which contact the motor neurons/axons with the compounds capable of binding markers of these cell types. In this regard the zebrafish may be dead or alive and, in order to visualise the labelled motor neurons/axons, it may be preferable to prepare sections of the zebrafish for use in microscopy. Sections of transgenic zebrafish for use in microscopy techniques detailed herein may also be preferred.

Antibodies and/or other compounds capable of binding such markers may be labelled with a detectable substance and one of skill will be familiar with the chemiluminescent (Alkaline phoshatase and HRP) and/or fluorescent compounds (FITC etc.) which may be used. Antibodies for use in this invention may be used in immunohistochemical techniques to label motor neuron/axons in zebrafish.

Each of the methods described above also incorporates the term “contacting” and the techniques which may be used to contact a test agent with a zebrafish are well known to one of skill in this field. Such techniques may include, for example, the injection of the agent into the zebrafish or yolk during adult and/or early embryonic stages. Additionally or alternatively, the test agent may be injected directly into certain cells, tissues, organs, structures or cells and/or administered by electroporation, canulation of the bladder/intestines, coating to a carrier composition and/or inclusion in porous beads. In a further embodiment, a test agent may be administered by adding it to food consumed by the zebrafish or to some other substrate that the zebrafish ingests and/or breathes. The compound may be injected into, for example, the developing embryo at the single cell stage, into the blood island cells or into the tail region. Additionally, or alternatively, the test agent may be added to the water in which the organism bathes such that when respiring and or feeding, the transparent non-human organism takes in the compound.

In addition, the techniques used to contact the test agent to the zebrafish described herein may be subjected to a protocol to improve the solubility in water. Such protocols may include the use of compounds such as DMSO or (2-hydroxypropyl)-beta-cyclodextrin)

It is to be understood that the term “test agent” should be taken to include (but not to be limited to) molecules, for example small organic molecules, proteins (such as antibodies and/or fragments thereof), peptides, amino acids, glycopeptides and nucleic acids including DNA, RNA and/or plasmids and/or antisense and/or inhibitory nucleic acids derived from either. Other compounds which may be subjected to the methods of the present invention may also include nucleic acid mimetics, such as, for example, morpholinos or PNAs and/or monosaccharides and/or polysaccharides.

One of skill will be familiar with commercially available compound libraries which may provide a source of test agents to be used in the methods described herein. By way of example, the test agents may be derived from the Spectrum Collection of FDA approved drugs etc., the Diversity Set of the US National Cancer Institute, the Tocriscreen library, the Prestwick Chemical Library and LOPAC 1280 compound library provided by Sigma-Aldrich.

It is to be understood that the list of test agents provided above is not exhaustive and one of skill in the art would readily be able to determine those molecules/compounds not listed here but which may also be subjected to the methods described herein.

In view of the above, a particular embodiment of this invention provides a method of identifying compounds potentially useful in the treatment of motor neuron degenerative diseases (MNDD), said method comprising the steps of:

(a) contacting a test agent with an embryonic zebrafish and determining the modulatory effect of said test agent on the growth, development, differentiation and/or regeneration of the motor neurons/axons;

(b) identifying test agents capable of modulating the growth, development, differentiation and/or regeneration of the motor neurons/axons in the embryonic zebrafish of step (a);

(c) contacting said identified agents with later-stage embryonic zebrafish and determining the modulatory effect of these test agents on the growth, development, differentiation and/or regeneration of the motor neurons/axons;

(d) identifying test agents capable of modulating the growth, development, differentiation and/or regeneration of the motor neurons/axons in the embryonic zebrafish of step (c); and

(e) contacting test agents identified in step (d) with an adult zebrafish having a spinal lesion and determining the modulatory effect of these test agents on the growth, development, differentiation and/or regeneration of the motor neurons/axons;

wherein test agents identified in step (e) as being capable of modulating the growth, development, differentiation and/or regeneration of the motor neurons/axons are potentially useful in the treatment of MNDD.

In one embodiment, step (a) of the above described method is conducted in accordance with the primary screen detailed above. In a further embodiment step (c) is conducted in accordance with the secondary screen detailed above. In a yet further embodiment, step (e) is conducted in accordance with the validation screen provided by the first aspect of this invention.

It should be understood that while it is beneficial to conduct all three screens (i.e. primary screen, secondary screen and validation screen) when identifying agents potentially useful in the treatment of MNDD, the invention should not be construed as being limited in this way. It is possible to use any of the methods described herein either in isolation or in combination in order to identify agents potentially useful in the treatment of MNDD.

In accordance with the above, the embryonic zebrafish used in step (a) may be the HB9:GFP transgenic zebrafish described above, the later-stage embryonic zebrafish used in step (c) may be the islet-1 transgenic zebrafish described above and the and the adult zebrafish may be the HB9:GFP, islet-1:GFP and/or the olig2:GFP transgenic fish also described above (and in the detailed description section below).

In addition to the primary, secondary and/or validation screens described above, test agents identified as being potentially useful in the treatment of MNDD (because of their modulatory effect on the growth, development, differentiation and/or regeneration of motor neurons/axons) may be further tested in an animal model to determine their suitability for use a therapeutic agents. Animal models may be designed to replicate the symptoms or pathology of particular diseases and/or conditions and in this regard should exhibit symptoms or pathology associated with any of the MNDD described herein. By way of example, rodent, for example rat, mouse, guinea pig and/or rabbit models of MNDD such as amyotrophic lateral sclerosis (ALS), Parkinson's disease, Alzheimer's disease may be particularly useful. In other embodiments, animals in which spinal cord lesions have been introduced, may be useful. One of skill will appreciate that compounds having therapeutic benefit will alleviate, reduce or cure the symptoms exhibited by the animal model.

An exemplary animal model in which transgenic mice overexpress variants of human mutations in superoxide dismutase 1 (SOD1) gene to replicate the symptoms of ALS, is provided by Puttaparthi et al., 2002 (Disease progression in a transgenic model of familial amyotrophic lateral sclerosis is dependant on both neuronal and non-neuronal zinc binding proteins. J Neurosci 22:8790-8796).

In a second aspect, the present invention provides compounds identified by any of the methods described herein for use in treating MNDD.

In a third aspect, the present invention provides the use of compounds identified by any of the methods described herein for the preparation of a medicament for treating MNDD.

In a fourth aspect, the present invention provides a method of treating a MNDD, said method comprising the step of administering to a patient suffering from a MNDD a therapeutically effective amount of a compound identified by any of the methods described herein.

In a fifth aspect, the present invention provides a pharmaceutical composition comprising one or more compounds identified by the methods described herein for use in treating a MNDD, in association with a pharmaceutically acceptable excipient, carrier or diluent.

Preferably, the pharmaceutical compositions provided by this invention are formulated as sterile pharmaceutical compositions. Suitable excipients, carriers or diluents may include, for example, water, saline, phosphate buffered saline, dextrose, glycerol, ethanol, ion exchangers, alumina, aluminium stearate, lecithin, serum proteins, such as serum albumin, buffer substances such as phosphates, glycine, sorbic acid, potassium sorbate, partial glyceride mixtures of saturated vegetable fatty acids, water salts or electrolytes, such as protamine sulphate, disodium hydrogen phosphate, potassium hydrogen phosphate, sodium chloride, zinc salts, colloidal silica, magnesium trisilicate, polyvinyl pyrrolidone, cellulose-based substances, polyethylene glycon, sodium carboxymethylcellulose, polyacrylates, waxes, polyethylene-polypropylene-block polymers, polyethylene glycol and wool fat and the like, or combinations thereof.

Said pharmaceutical formulation may be formulated, for example, in a form suitable for topical, parenteral (injectable) or oral administration. For example the formulation for topical administration may be presented as an ointment, solution or a suspension in an aqueous or non-aqueous liquid, or as an oil-in-water liquid emulsion.

In this regard, the inventors have used the methods described herein and have identified compounds which may be useful in the treatment of MNDDs. As such, the present invention, and in particular the second-fifth aspects of this invention relate to the following compounds:

(i) norapomorphine

(ii) apomorphine

(iii) cycloheximide

(iv) taxol

(v) mavastatin

(vi) 13-cis retinoic acid

(vii) methotrexate

or derivatives, homologues or variants thereof.

In addition, the inventors have discovered that compounds which exhibit dopamine agonist activity may be useful as compounds, medicaments or compositions for treating MNDDs (or as part of a treatment regime or method for treating the same). More specifically, compounds which agonise the dopamine receptors, for example those belonging to the D₁ receptor-like family and those belonging to the D₂ receptor-like family. More specifically, useful compounds may agonise the D₂ and/or D₄ dopamine receptors.

Suitable examples may include compounds having the formula:

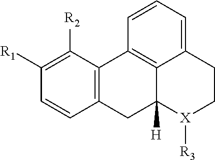

or a physiologically acceptable salt, solvate, ester or amide thereof,

wherein

X represents oxygen sulphur, or NH, or N when R₃ is present;

R₁ and R₂ are independently selected from the group consisting of hydrogen, halogen, nitro, cyano amide, hydroxyl, substituted or unsubstituted alkyl, substituted or unsubstituted alkenyl, substituted or unsubstituted aryl or heteroaryl, substituted or unsubstituted aralkyl, alkoxy, amino, mono- or di-alkyl substituted amino, sulphydryl, formyl, carboxyl, carboxylic acid, sulphonate, sulphonic acid, quaternary ammonium, C(═O)OR₄, C(═S)OR₄, C(═O)SR₄, C(═S)SR₄, C(═O)NH₂ and C(═S)NH₂ wherein one or both hydrogen atoms may be independently exchanged for R₄; and

R₃ and R₄ when present are each independently selected from the group consisting of substituted or unsubstituted alkyl, substituted or unsubstituted alkenyl, substituted or unsubstituted alkynyl, and substituted or unsubstituted —(CH₂)ₙ-aryl, wherein n is a number from 0 to 10.

In one embodiment, R₁, R₂ may be hydroxy or alkoxy. In further embodiments, R₁, R₂ may be both hydroxy. Additionally, or alternatively, R₃ may be linear or branched alkyl. In other embodiments, R₃ may be a linear or branched alkyl of from 1 to 6 carbons. R₃ may be propyl, in particular n-propyl.

Alkyl groups may be linear cyclic or branched. Typically alkyl groups will comprise from 1 to 25 carbon atoms, more usually 1 to 10 carbon atoms, more usually still 1 to 6 carbon atoms.

Alkyl, alkenyl, alkynyl and aryl groups may be substituted, for example once, twice, or three times, e.g. once, i.e. formally replacing one or more hydrogen atoms. Examples of such substituents are halo (e.g. fluoro, chloro, bromo and iodo), aryl, heteroaryl, hydroxy, nitro, amino, alkoxy, alkylthio, carboxy, cyano, thio, formyl, ester, acyl, thioacyl, amino, carbamido, sulfonamido and the like. Examples of aryl and heteroaryl substituted alkyl include CH₂-aryl (e.g. benzyl) and CH₂-heteroaryl.

In one embodiment, a compound useful in the treatment of MNDDs may have the formula:

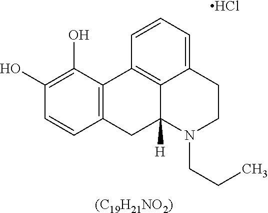

Compounds of this type may be known as R(−)-Propylnorapomorphine (NPA), and pharmaceutically acceptable salts such as R(−)-Propylnorapomorphine hydrochloride may be particularly useful in the treatment of MNDD.

As such, the invention may be taken to relate to the methods, compositions, medicaments and compounds selected from the group consisting of norapomorphine, apomorphine, cycloheximide, taxol, mavastatin, I 3-cis retinoic acid, methotrexate and NPA (i.e. R(−)-Propylnorapomorphine: as described above) for use in treating MNDD.

The term MNDD should be taken to include any disease or disorder in characterized by the degeneration, loss or damage or motor neurons/axons and may include, for example, diseases such as amyotrophic lateral sclerosis (ALS), Parkinson's disease, Alzheimer's disease. The term MNDD may also encompass the loss or damage to motor neurons/axons occurring as a result of spinal cord injury.

### DETAILED DESCRIPTION

The present invention will now be described in detail and with reference to the following Figures which show:

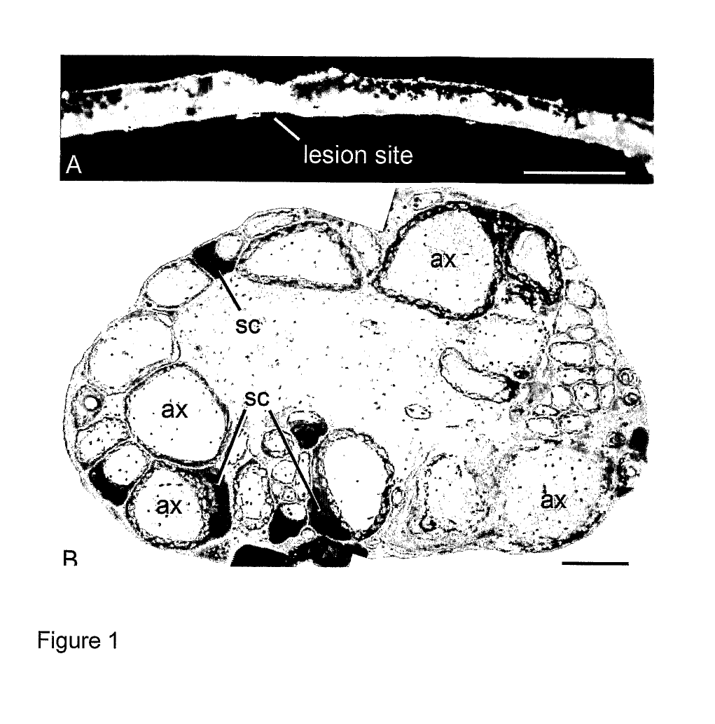

FIG. 1: The lesion site consists mainly of regenerated axons. A: A lateral stereo-microscopic view of a dissected spinal cord is shown (rostral is left). The dorsal aspect of the spinal cord is covered by melanocytes and the tissue bridging the lesion site appears translucent. B: An electron-microscopic cross section through the lesion site is shown. The lesion site consists mainly of axons (ax), some of which are re-myelinated by Schwann cells (sc). Bar in A=1 mm, in B=5 μm.

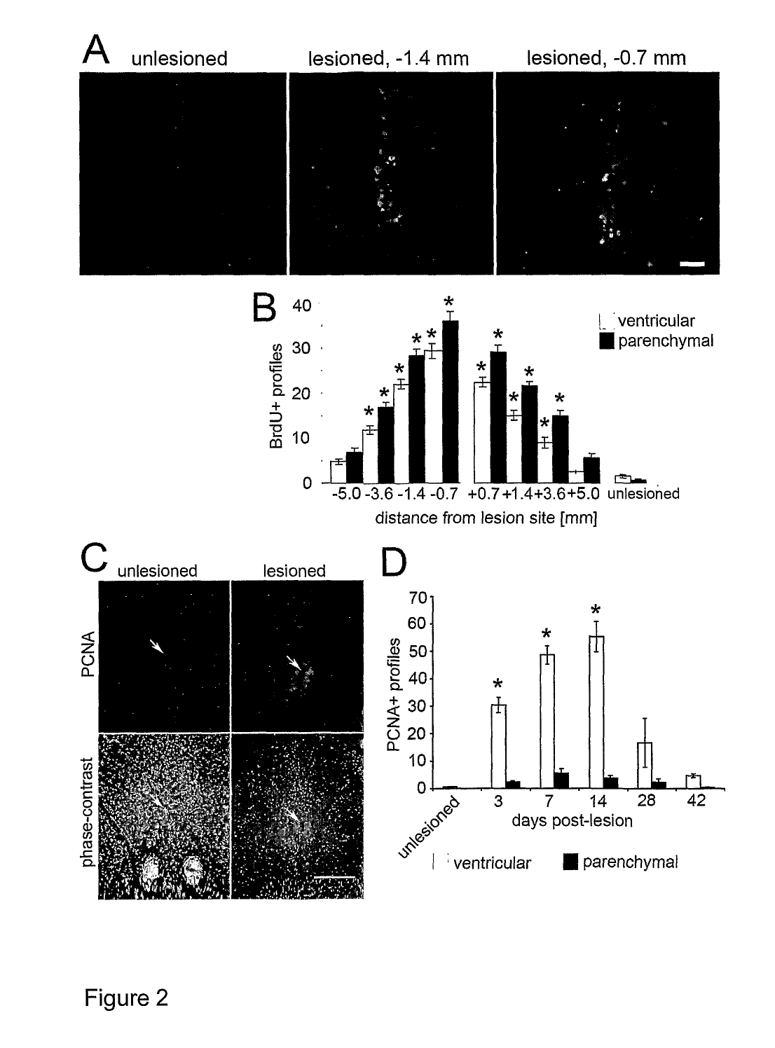

FIG. 2: Lesion-induced proliferation in the adult spinal cord. Confocal images of spinal cross sections are shown (dorsal is up). A: BrdU labeling of spinal cross sections shows a massive increase in labeling in the ventricular zone at 14 days post-lesion (injections 0, 2, and 4 days post-lesion). The highest density of BrdU⁺ cells is detectable in the ventricular zone close to the lesion site. B: Quantification of BrdU⁺ profiles at 14 days post-lesion indicates significant proliferative activity up to 3.6 mm rostral and caudal to the lesion epicenter (n=3 animals per treatment, p<0.0001). Even though the non-ventricular area of spinal cross sections is much larger than that of the ventricular zone, only slightly more proliferating cells were observed in the non-ventricular area, indicating a high density of labeled cells in the ventricular zone. C: PCNA immunohistochemistry indicates a strong increase in the number of proliferating cells in the ventricular zone (arrows) at 14 days post-lesion. D: The number of proliferating ventricular, but not parenchymal cell profiles/section was significantly increased after a lesion and peaked at 14 days post-lesion (n=3 animals per time point, p<0.0001). Bar in A=25 μm, in C=50 μm.

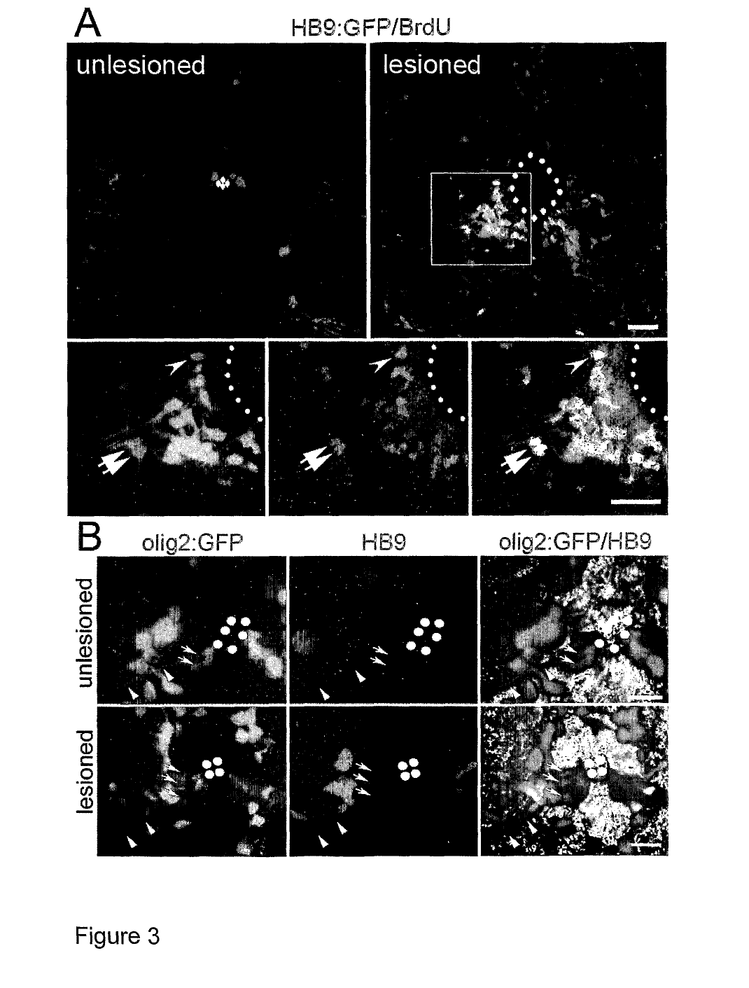

FIG. 3: Generation of new motor neurons in the lesioned spinal cord. Confocal images of spinal cross sections at 2 weeks post-lesion are shown (dorsal is up). A: HB9:GFP⁺/BrdU⁺ neurons are present in the lesioned, but not the unlesioned vetral horn. These cells (boxed in upper right and shown in higher magnification in bottom row) bear elaborate processes (arrows) or show ventricular contact (arrowhead). Dots outline the ventricle. B: Olig2:GFP⁺ progenitor cells (arrows) have long radial processes (arrowheads), contact the ventricle (outlined by dots), and are double-labeled with an HB9 antibody at 2 weeks post-lesion, but not in the unlesioned spinal cord. Bars in A=25 μm; bars in B=7.5 μm (upper row), 15 μm (lower row).

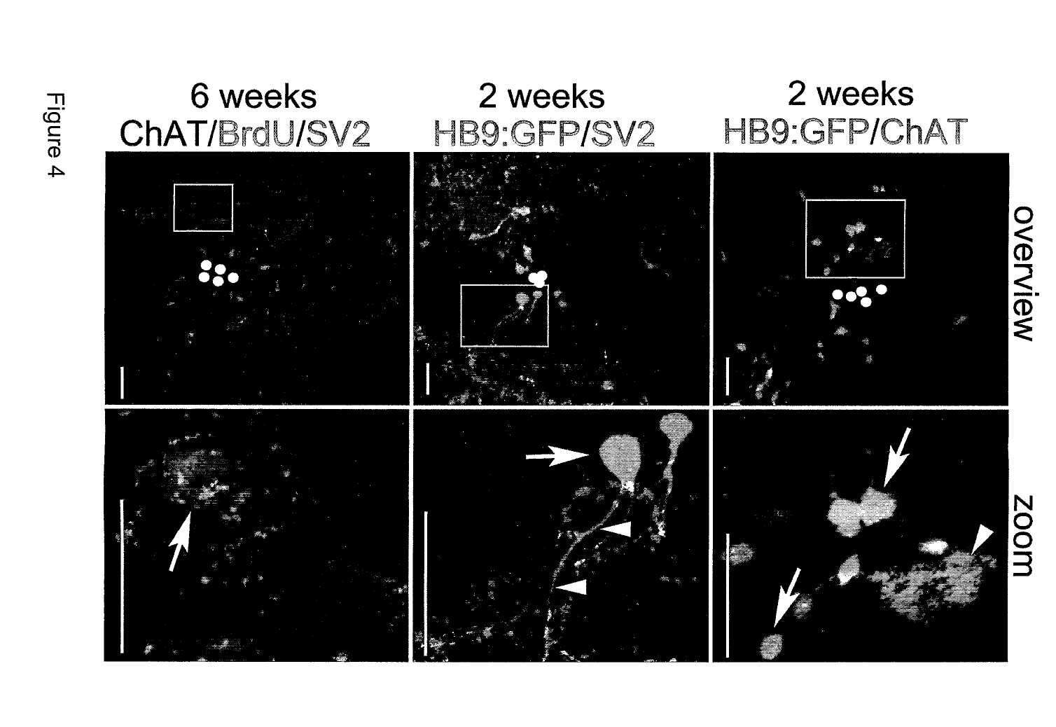

FIG. 4: Maturation of newly generated motor neurons. Confocal images of spinal cross sections are shown (dorsal is up). Clusters of newly generated HB9:GFP⁺ motor neurons are ChAT-(arrows A; arrowhead indicates a ChAT⁺/HB9:GFP⁻ differentiated motor neuron). Somata (arrow B) and proximal dendrites (arrowheads B) receive few SV2⁺ contacts at 2 weeks post-lesion. At 6 weeks post-lesion, ChAT⁺/BrdU⁺ somata are decorated with SV2⁺ contacts (arrow C). Inset (right panel C) depicts a ChAT⁺ motor neuron that is decorated with SV2⁺ contacts in an unlesioned animal. At 8 weeks post-lesion, a BrdU+ cell is retrogradely traced from the muscle tissue (D). A-C: bars=25 μm; D: bars=15 μm.

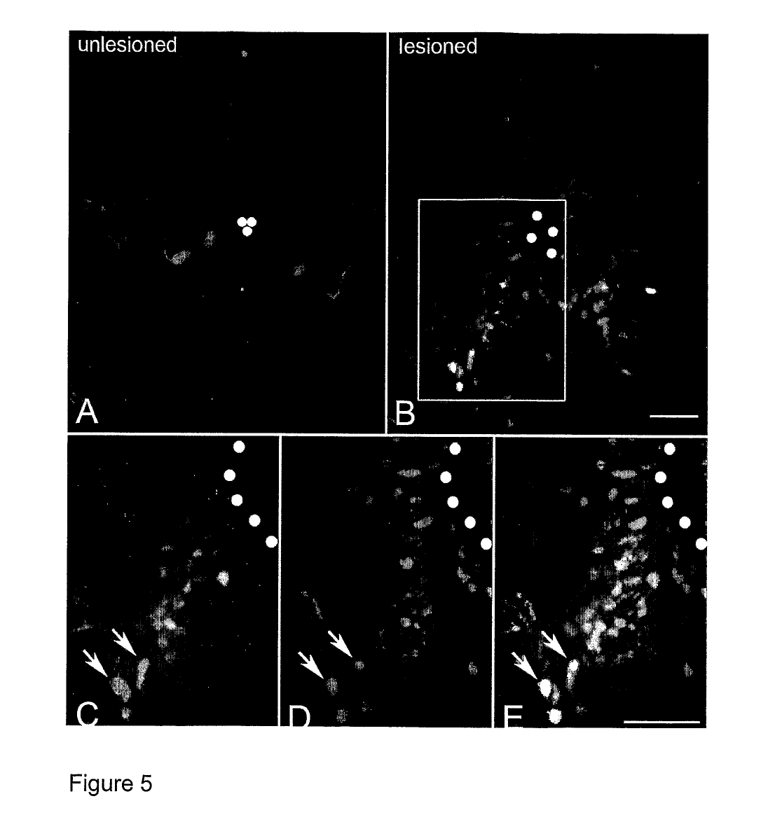

FIG. 5: Newly generated small islet-1:GFP⁺ cells in the lesioned spinal cord. Cross sections through the spinal cord of unlesioned (A) and lesioned (B-E) animals at 2 weeks post-lesion are shown. In unlesioned animals, only large GFP⁺ cells are detectable, whereas many smaller GFP⁺ cells are present in the ventral horn of the lesioned spinal cord. Many of these cells are also BrdU⁺, as indicated by arrows in the higher magnification (C-E) of the area boxed in B. Dots outline the ventricle. Bars=25 μm.

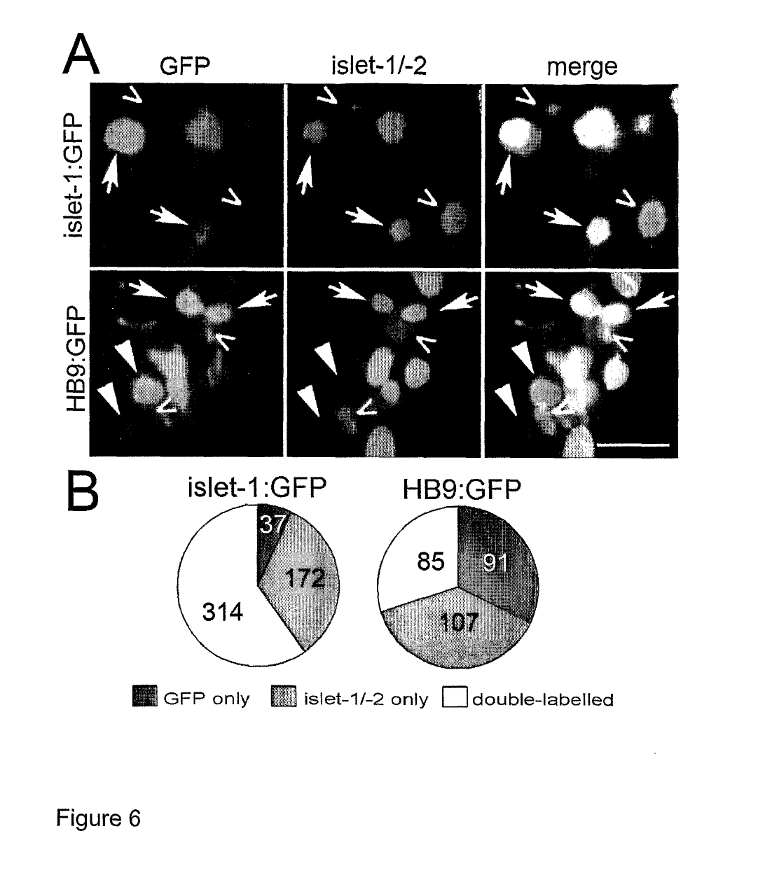

FIG. 6: Islet-1/-2 immunohistochemistry and transgenic motor neuron markers partially overlap in the lesioned spinal cord. A: Islet-1:GFP⁺ cells are double-labeled by the islet-1/-2 antibody, confirming specificity of transgene expression. A substantial proportion of HB9:GFP⁺ cells are not double-labeled by the antibody and many cells are only labeled by the islet-1/-2 antibody in both transgenic lines, indicating that the marker profiles of newly generated motor neurons are heterogeneous after a lesion. Arrows indicate double-labeled neurons, arrowheads indicate neurons only labeled by the transgene and open arrowheads point to cells only labeled by the antibody. B: Summations of all cells counted in 6 sections (50 μm thickness) per animal from the region of 1.5 mm surrounding the lesion site (n=3 animals for each transgene) are indicated. The small proportion of cells only labeled by GFP in islet-1:GFP animals may result from higher stability of the GFP than endogenous islet-1 detected by the islet-1/-2 antibody. Bar=25 μm.

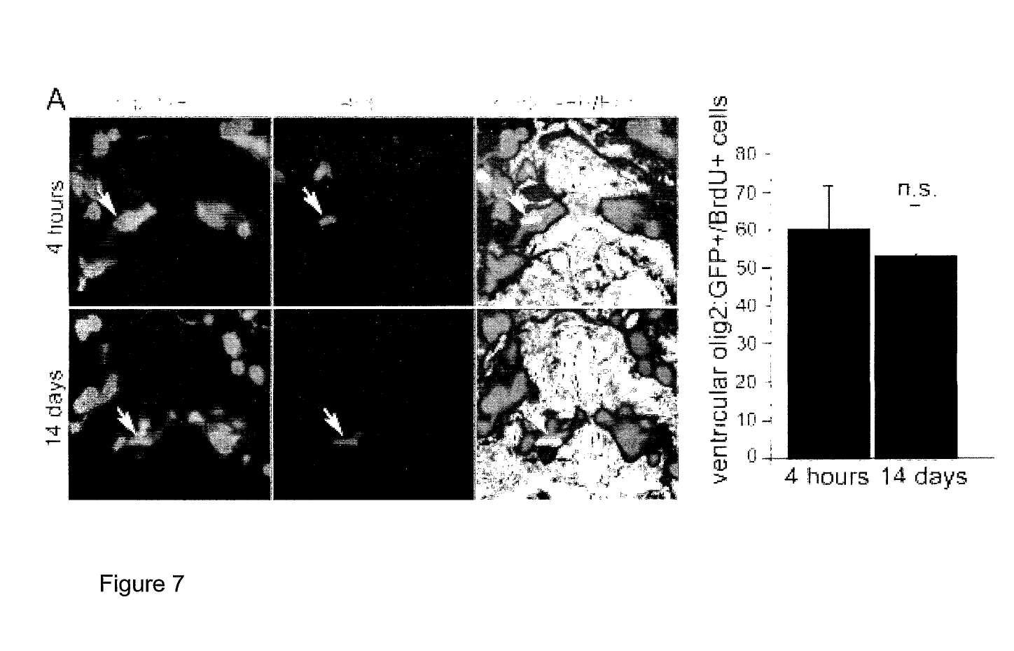

FIG. 7: Label retention in olig2:GFP ependymo-radial glial cells. A: A subpopulation of olig2:GFP⁺ ependymo-radial glial cells is BrdU⁺ at 4 hours and 14 days after a single application of BrdU at 14 days post-lesion. Bar=15 μm. B: No significant differences in the number of olig2:GFP⁺/BrdU⁺ cells were observed between both time points of analysis.

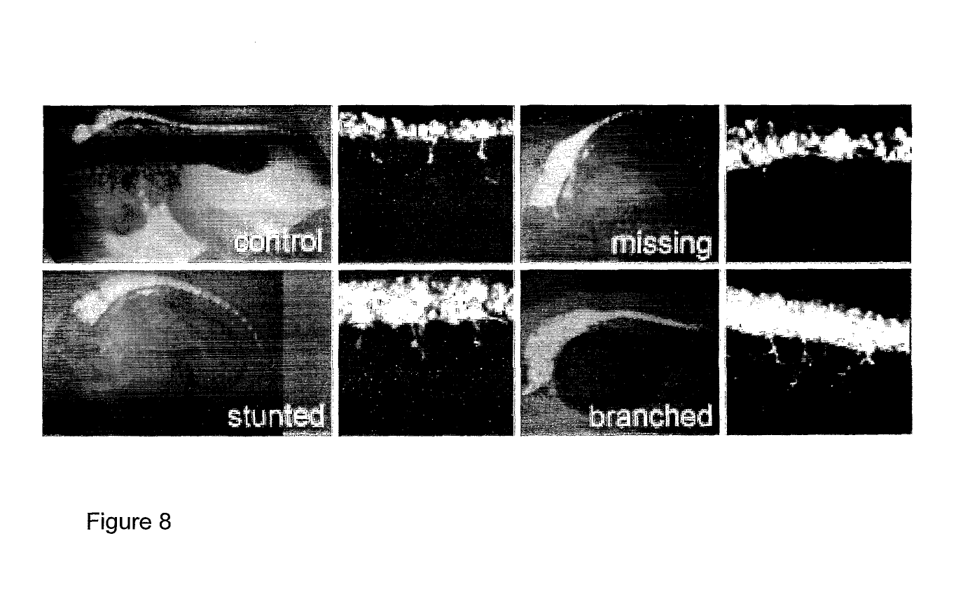

FIG. 8: The primary screening paradigm. Whole embryos and axonal phenotypes are shown. Applying compounds to HB9:GFP embryos at 6 hpf leads to different types of aberrations of motor axons at 24 hpf. Note that embryos show different degrees of malformations, such that non-specific effects on axonal morphology cannot be excluded.

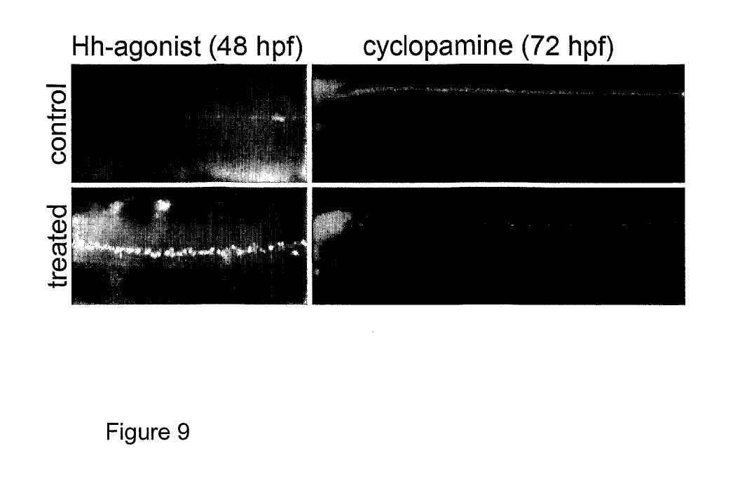

FIG. 9: The secondary screening paradigm. Lateral views of the trunk are shown. In islet-1:GFP transgenic fish, application of a hedgehog agonist leads to premature differentiation of a subclass of motor neurons in the ventral spinal cord at 48 hpf. At 72 hpf, when these neurons are present in control animals, cyclopamine blocks their differentiation. The right column shows pictures of live embryos, taken with a camera-equipped stereo-microscope. (On the far left, strong expression in hindbrain neurons is visible).

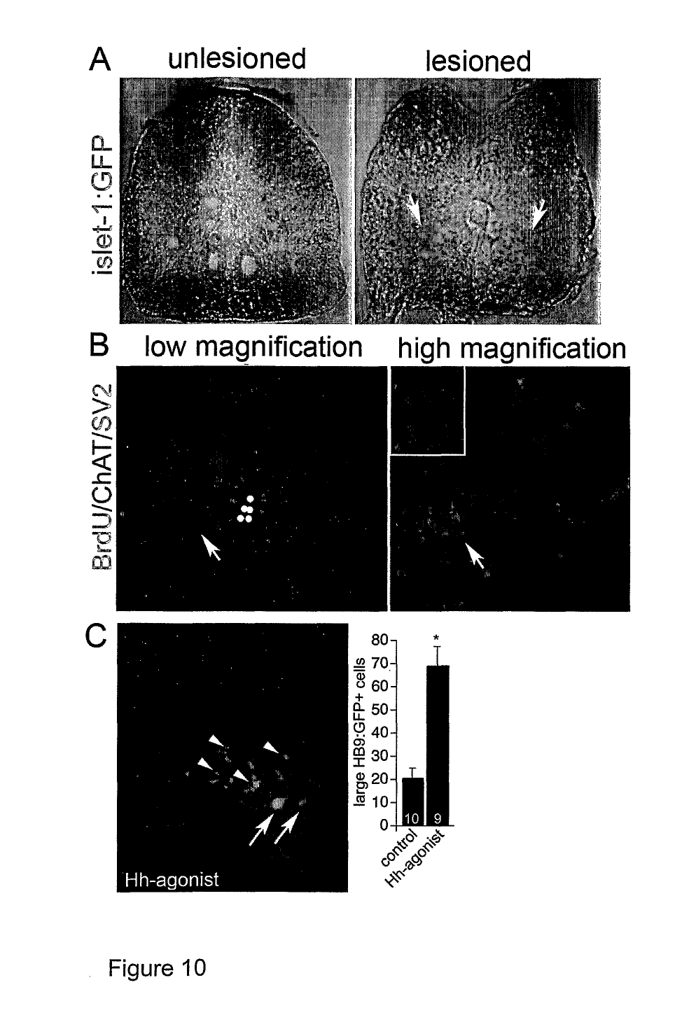

FIG. 10: The adult regeneration paradigm. Cross sections of the adult spinal cord are shown. A: In unlesioned adult islet-1:GFP fish only few large motor neurons are present. These disappear after a lesion, but many newly generated small motor neurons (arrows) appear at 2 weeks post-lesion. B: At 6 weeks post-lesion, newly generated (BrdU+), fully differentiated (ChAT+) motor neurons are present that are decorated by synapse-like contacts (SV2+), suggesting integration of these new motor neurons (arrow) into the spinal circuitry (dots outline the ventricle). Inset shows a motor neuron in an unlesioned fish. C: Intraperitoneal injection of a Hh-agonist trebles the number of large differentiated motor neurons (arrows). Many small newly generated motor neurons are also present (arrowheads).

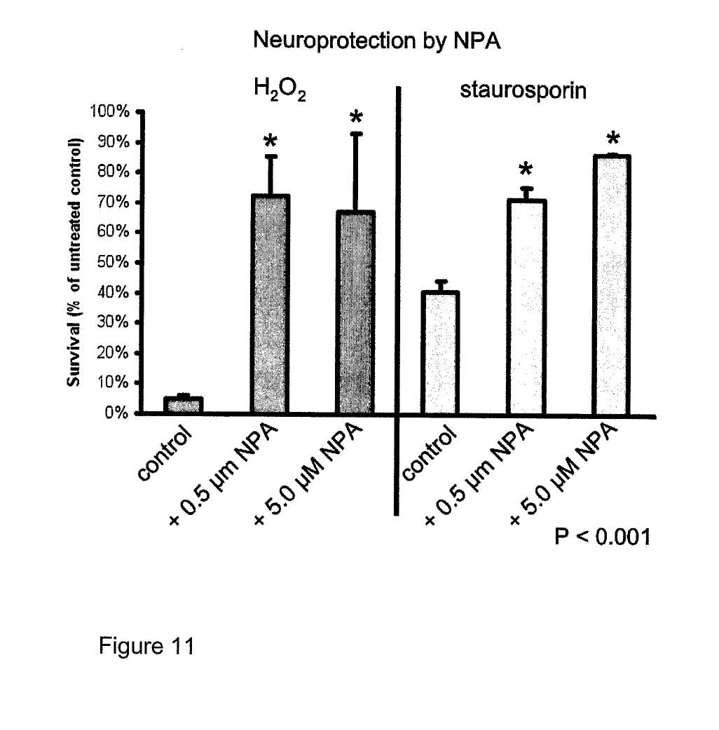

FIG. 11: Shows the results of an assay investigating the neuroprotective effect of NPA on primary mammalian motor neurons in culture. Neuroprotection is measured as a % of cells counted in each experimental condition to the number of control, untreated cells run at the same time on the same plate. NPA was applied 1 hr before stressors (H₂O₂ and staurosporin), at 2 concentrations: 0.5 and 5 μM. Stressors were applied for 24 hours, then plates were fixed and stained for MAP-2 and then positive cells counted. Results clearly show that NPA significantly increases the number of mammalian neurons surviving stress conditions.

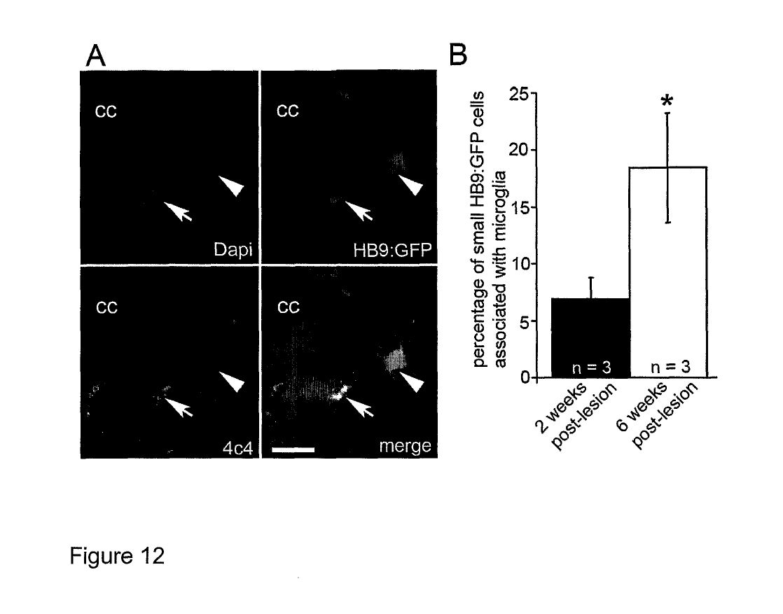

FIG. 12: An increasing percentage of small Hb9:GFP⁺ cells is associated with macrophages/microglial cells after a lesion. A: A HB9:GFP⁺ cell that is associated with macrophage/microglial cell marker 4c4 (arrow) and a HB9:GFP⁺ cell that is not associated with 4c4 (arrowhead) is depicted. Bar=10 μm. B: A higher percentage of HB9:GFP⁺ cells is associated with 4c4 immuno-reactivity at later time points of regeneration (p=xx). Density of 4c4-positive cells was comparable between the two time points (data not shown).

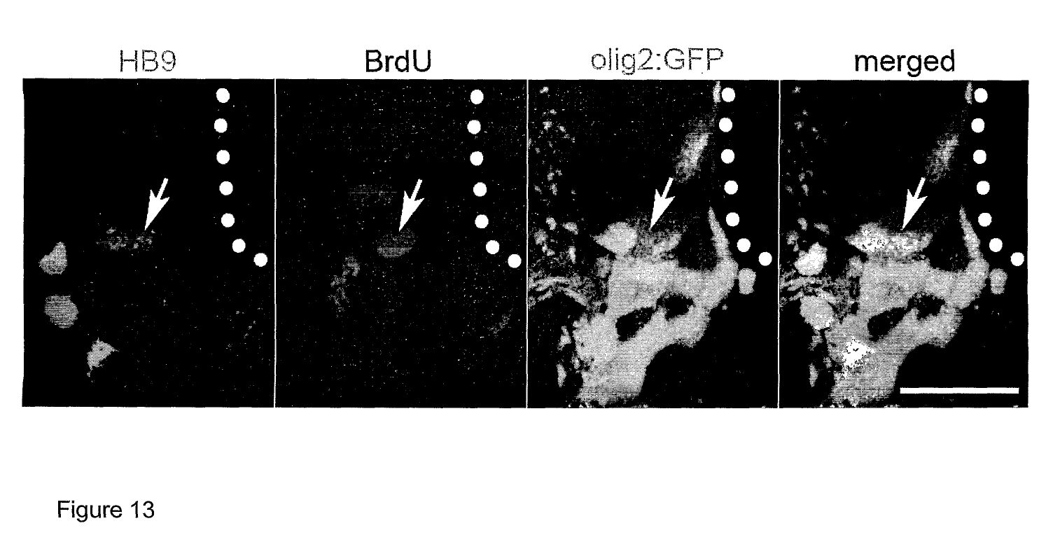

FIG. 13: HB9/BrdU/olig2:GFP triple labeled cells are found in the ventro-lateral ventricular zone. Arrow indicates a triple labeled cell. Dots outline the ventricle. Bar=25 μm.

### MATERIALS AND METHODS

### Animals

All fish are kept and bred in our laboratory fish facility according to standard methods (Westerfield, 1989) and all experiments have been approved by the British Home Office. We used wild type (wik), HB9:GFP (Flanagan-Steet et al., 2005), islet-1:GFP (Higashijima et al., 2000) and olig2:GFP (Shin et al., 2003) transgenic fish. Consistency of transgene expression with that of the endogenous genes at the adult stage was verified by immunohistochemistry (HB9 and islet-1, FIG. 6 and not shown) or in situ hybridization (olig2, not shown) for the respective genes.

### Spinal Cord Lesion

As described previously (Becker et al., 1997), fish were anesthetized by immersion in 0.033% aminobenzoic acid ethylmethylester (MS222; Sigma, St. Louis, Mo.) in PBS for 5 min. A longitudinal incision was made at the side of the fish to expose the vertebral column. The spinal cord was completely transected under visual control 4 mm caudal to the brainstem-spinal cord junction.

### Electron Microscopy

Ultrathin sections (75-100 nm in thickness) were prepared and observed by electron microscopy as published previously (Becker et al., 2004).

### Immunohistochemistry

We used rat anti-BrdU (BU 1/75, 1:500, AbD Serotec, Oxford, UK), mouse anti-islet-1/-2 (Tsuchida et al., 1994) (40.2D6, 1:1000, Developmental Studies Hybridoma Bank, Iowa City, USA), mouse anti-HB9 (MNR2, 1:400, Developmental Studies Hybridoma Bank) mouse anti-PCNA (PC10, 1:500, Dako Cytomation, Glostrup, Denmark) and goat anti-ChAT (AB144P, 1:250, Chemicon, Temecula, USA) antibodies. Secondary Cy3-conjugated antibodies were purchased from Jackson ImmunoResearch Laboratories Inc. (West Grove, Pa., USA). Animals were transcardially perfused with 4% paraformaldehyde and post-fixed at 4° C. overnight. Spinal cords were dissected, embedded in 4% agar and sectioned (50 μm thickness) with a vibrating blade microtome (Microm, Volketswil, Switzerland). Antigen retrieval was carried out by incubating the sections for 1 hour in 2 M HCl for BrdU immunohistochemistry, or by incubation in citrate buffer (10 mM sodium citrate in PBS, pH=6.0) at 85° C. for 30 minutes for HB9, islet-1/-2 and PCNA immunohistochemistry. All other steps were carried out in PBS (pH 7.4) containing 0.1% triton-X100. Sections were blocked in goat or donkey serum (15 μl/ml) for 30 minutes, incubated with the primary antibody at 4° overnight, washed three times 15 minutes, incubated with the appropriate secondary antibody for 1 h, washed again, mounted in 70% glycerol and analyzed using a confocal microscope (Zeiss Axioskop LSM 510). Double-labeling of cells was always determined in individual confocal sections. Immunohistochemistry on 14 μm cryosections was performed as described (Becker and Becker, 2001).

### Intraperitoneal BrdU Application

Animals were anaesthetised and intraperitoneally injected. We injected 5-bromo-2-deoxyuridine (BrdU, Sigma-Aldrich, UK) solution (2.5 mg/ml) at a volume of 50 μl at 0, 2, 4 days post-lesion for most experiments.

### Retrograde Axonal Tracing

Motor neurons in the spinal cord were retrogradely traced by bilateral application of biocytin to the muscle periphery at the level of the spinal lesion, as previously described (Becker et al., 2005), with the modification that biocytin was detected with Cy3-coupled streptavidin (Molecular Probes, Eugene, Oreg., USA) in spinal sections. This was followed by immunohistochemistry for BrdU (see above).

### Cell Counts and Statistical Analysis

Stereological counts were performed in confocal image stacks of three randomly selected vibratome sections from the region up to 750 μm rostral to the lesion site and three sections from the region up to 750 μm caudal to the lesion site. Cell numbers were then calculated for the entire 1.5 mm surrounding the lesion site.

PCNA⁺ and BrdU⁺ nuclear profiles in the ventricular zone (up to one cell diameter away from the ventricular surface) were determined in vibratome sections (50 μm thickness) in the same region of spinal cord. At least 6 sections were analyzed per animal by fluorescence microscopy and values were expressed as profiles per 50 μm section. The observer was blinded to experimental treatments. Variability of values is given as standard error of the mean. Statistical significance was determined using the Mann-Whitney U-test (p<0.05) or ANOVA with Bonferroni/Dunn post-hoc test for multiple comparisons.

### Results

### A Spinal Lesion Induces Wide-Spread Ventricular Proliferation.

To determine the spinal region in which new motor neurons might regenerate, we analyzed the overall organization of the regenerated spinal cord. At 6 weeks post-lesion, when functional recovery is complete (Becker et al., 2004), the lesion site itself had not restored normal spinal architecture and consisted mainly of unmyelinated and re-myelinated regenerated axons (FIG. 1). Immediately adjacent to this axonal bridge, spinal cross sections showed normal cytoarchitecture, with the exception that white matter tracts were filled with myelin debris of degenerating fibers (Becker and Becker, 2001). This indicated that this tissue existed before the lesion was made. Thus, no significant regeneration of whole spinal cord tissue occurred for up to at least 6 weeks post-lesion.

To find newly generated cells in the unlesioned and lesioned spinal cord, we used immunohistochemical detection of repeatedly injected (0, 2, and 4 days post.-lesion) 5-bromo-2-deoxyuridine (BrdU), which labels cells that have divided. This revealed that very few cells proliferated in the unlesioned spinal cord. At 2 weeks post-lesion, we observed a significant increase in the number of newly generated cells in the spinal tissue up to 3.6 mm rostral and caudal to the lesion site, covering more than a third of the length of the entire spinal cord. BrdU⁺ labeled cells were found throughout spinal cross sections, but appeared to be concentrated at the midline and in the ventricular zone around the central canal (FIG. 2A). Numbers of newly generated cells were highest close to the lesion site (FIG. 2B).

To localize acutely proliferating cells in the spinal cord, we used immunohistochemistry with the PCNA antibody, which labels cells in early G1 phase and S phase of the cell cycle. This revealed a significant increase in proliferating cells solely in the ventricular zone. Proliferation peaked at 2 weeks post-lesion and had returned to values that were similar to those of unlesioned animals by 6 weeks post-lesion (FIG. 2C,D).

### Numbers of Differentiating Motor Neurons are Dramatically Increased in the Lesioned Zebrafish Spinal Cord.

We determined whether new motor neurons are generated in the core region of ventricular proliferation comprising 1.5 mm surrounding the lesion site. We examined the numbers of cells expressing green fluorescent protein (GFP) in transgenic lines, in which GFP expression labels motor neurons under the control of the promoters for HB9 (Flanagan-Steet et al., 2005) or islet-1 (Higashijima et al., 2000). In unlesioned HB9:GFP animals, few large (diameter>12 μm) motor neurons and very few smaller (diameter<12 μm) GFP⁺ motor neurons (20 cells±7.7, n=4 animals) were observed in the ventral horn. The number of small GFP⁺ cells was non-significantly increased at 1 week post-lesion (207±84.5 cells; n=3 animals; p=0.3), but was massively increased at 2 weeks post-lesion (870±106.8 cells, n=11 animals; p=0.004; FIG. 3A). Similar observations were made in islet-1:GFP animals (Table 1, FIG. 5). Values for HB9:GFP⁺ small motor nerons were significantly reduced by 6 to 8 weeks post-lesion (251±78.7 cells, n=6 animals). Although still elevated, these values were not significantly different from those in unlesioned animals anymore (p=0.2, Table 1). Double-labeling with a macrophage/microglial marker and Terminal Transferase dUTP Nick End Labeling (TUNEL) suggested that many of the cells died between 2 and 6 to 8 weeks post-lesion (FIG. 12). Immunohistochemistry for HB9 and islet-1/-2 proteins confirmed the time course of motor neuron numbers and indicated that increases in motor neuron numbers were strongest in the vicinity of the lesion site (FIG. 6 and data not shown). Thus, numbers of differentiating motor neurons significantly increased after a spinal lesion.

Double-labeling of islet-1/-2 antibodies in HB9:GFP and islet-1:GFP transgenic animals revealed that motor neurons were heterogeneous in marker expression (FIG. 6). This suggests that, similar to development (Tsuchida et al., 1994; William et al., 2003), islet-1/-2 and HB9 expression diverged in spinal motor neurons depending on differentiation stage and subtype of motor neuron.

To directly show that new motor neurons were generated after a lesion, we repeatedly injected animals with BrdU at 0, 2, and 4 days post.-lesion. In the unlesioned spinal cord we observed no double-labeled motor neurons in islet-1:GFP animals (n=5 animals) and only one cell so labeled in HB9:GFP animals (n=4). Even an extended BrdU injection protocol (injections at days 0, 2, 4, 6, 8, analysis at day 14) did not yield any HB9:GFP⁺/BrdU⁺ cells in unlesioned fish (n=5 animals). Since the bio-availability of BrdU is approximately 4 hours post-injection (Zupanc and Horschke, 1995), we cannot exclude a very low proliferation rate of motor neurons. However, we do not find any evidence to indicate substantial motor neuron generation in the unlesioned mature spinal cord.

In contrast, in lesioned HB9:GFP animals, 200±46.2 cells (n=7 animals, p=0.0076) and in islet-1:GFP animals, 184±49.3 cells (n=3 animals, p=0.0104) were double-labeled by the transgene and BrdU at 2 weeks post-lesion (FIG. 3A). Less than 8% of all BrdU+ cells were HB9:GFP⁺ (7.6%±1.86%) or islet-1:GFP+(6.3%±1.75%), suggesting proliferation of additional neuronal and non-neuronal cell types. Thus, a spinal lesion induces generation of new motor neurons and possibly other cell types in adult zebrafish.

New Motor Neurons are Likely Derived from Olig2 Expressing Ependymo-Radial Glial Cells.

Next, we analyzed expression of the transcription factor olig2, which is essential for motor neuron generation during development. In transgenic fish expressing GFP under the olig2 promoter, GFP is found in oligodendrocytes and in a ventro-lateral subset of ependymo-radial glial cells (FIG. 3B and Park et al., 2007). After a lesion, these cells proliferate, as indicated by double-labeling with PCNA at 2 weeks post-lesion. We found 490±224.2 PCNA+/olig2:GFP+ ependymo-radial glial cells and 217±103.8 non-ventricular PCNA+/olig2:GFP+ cells (n=2 animals) in the 1500 μm surrounding the lesion site at 2 weeks post-lesion. In situ hybridization for olig2 mRNA labeled parenchymal cells, as well as ependymal cells in the same ventricular position as olig2:GFP+ependymo-radial glial cells in the lesioned spinal cord (data not shown), indicating specificity of the transgenic label. These observations suggest that olig2 expressing cells could give rise to motor neurons during regeneration.

To analyze the relationship between olig2 expressing potential stem cells and motor neurons more directly we used immunohistochemistry for HB9 and islet-1/-2 in olig2:GFP transgenic animals. The relative stability of GFP, outlasting that of many endogenous proteins, has been used as a lineage tracer in transgenic fish to determine the progeny of adult retinal (Bernardos et al., 2007) and tegmental (Chapouton et al., 2006) progenitor cells. In unlesioned animals, no double-labeling of GFP and HB9 (n=3 animals) or GFP and islet-1/-2 (n=4 animals) was observed. At 2 weeks post-lesion, parenchymal olig2:GFP+ cells did not co-expressed either HB9 or islet-1/-2, which makes it unlikely that these cells gave rise to motor neurons. In contrast, a substantial subpopulation of olig2:GFP⁺ ependymo-radial glial cells were HB9⁺ (204±32.2 cells; n=3 animals; FIG. 3B) or islet-1/-2⁺ (34±8.9 cells; n=4 animals, not shown). Double-labeling indicated that either there was an overlap in the expression of olig2 and HB9 or islet-1/-2 during differentiation of motor neurons in the ventricular zone or recently differentiated HB9⁺ and islet-1/-2⁺ motor neurons had retained the GFP. Differences in the numbers of olig2:GFP+ ependymo-radial glial cells that expressed HB9 or islet-1/-2 may be related to differences in differentiation stage or subtype of motor neuron produced (William et al., 2003). If olig2:GFP⁺ ependymo-radial glial cells give rise to HB9⁺ motor neurons, we should be able to demonstrate that HB9⁺/olig2:GFP⁺ cells are newly generated. Indeed, 74±22.7 HB9⁺/olig2:GFP⁺ cells (n=3 animals) were labeled with BrdU (injections at 0, 2, and 4 days post.-lesion) at 14 days post-lesion in the 1500 μm surrounding the lesion site (FIG. 13). In the ventricular zone dorsal and ventral to the olig2:GFP⁺ region, expression of HB9 or islet-1/-2 was rarely observed, suggesting that olig2:GFP⁺ ependymo-radial glial cells were the main source of new motor neurons (FIG. 3B). Thus, olig2 expressing ependymo-radial glial cells switch to motor neuron production after a lesion.

To determine whether olig2:GFP⁺ ependymo-radial glial cells have stem cell characteristics we determined whether these cells retained BrdU label and were thus slowly-proliferating (Chapouton et al., 2006). Lesioned olig2:GFP animals were injected with a single pulse of BrdU at 14 days post-lesion and the number of olig2:GFP⁺/BrdU⁺ cells in the ventricular zone was assessed at 4 hours and 14 days post-injection. Numbers were not significantly different at the two time points (4 hours: 60±11.5 cells, n=5 animals; 14 days: 53±13.3 cells, n=4 animals, p=0.6), indicating that olig2:GFP⁺ cells did indeed retain label (FIG. 7). This suggests the possible presence of motor neuron stem cells among the population of olig2:GFP⁺ ependymo-radial glial cells. However, we cannot exclude that label-retaining cells give rise to other cell types.

### Evidence for Terminal Differentiation of New Motor Neurons

Newly generated small motor neurons were not fully differentiated at 2 weeks post-lesion. These cells were either attached to the ventricle with a single slender process, or were located farther away from the ventricle with several processes into the grey matter that were up to or greater than 100 μm long in HB9:GFP and in islet-1:GFP transgenic fish (FIGS. 3A, 4). However, even the cells with long processes showed very little apposition of somata and processes with SV2⁺ contacts, an indicator of synaptic coverage (FIG. 4B). Moreover, small HB9:GFP⁺ neurons rarely expressed ChAT (2.7%±0.90%, n=3 animals), a marker of mature motor neurons (Arvidsson et al., 1997), at 2 weeks post-lesion (FIG. 4A).

In contrast to small HB9:GFP⁺ cells, large HB9:GFP⁺ cells were mostly ChAT⁺ in unlesioned animals, (80.6%±6.92%, n=3 animals) indicating that these were fully differentiated motor neurons. At 1 (42±15.1 cells, n=3 animals, p=0.0035) and 2 weeks post-lesion (40±7.3 cells, n=11 animals, p<0.0003), large diameter HB9:GFP⁺ motor neurons were strongly reduced in number, compared to unlesioned animals (133±34.9 cells, n=4 animals). Similar observations were made in islet-1:GFP animals (Table 1). This suggests lesion-induced loss of motor neurons, which was confirmed by macrophage/microglia and TUNEL labeling of hb9:GFP⁺ motor neurons at 3 days post-lesion (FIG. 12). At 6 to 8 weeks post-lesion lesion, there was an increase in the number of large diameter HB9:GFP⁺ cells to 91±11.5 cells (n=6 animals), such that cell numbers were not different from those in unlesioned animals anymore (p=0.081). Large diameter ChAT⁺ cells showed similar dynamics (unlesioned: 478±111.1 cells, n=3 animals; 2 weeks post-lesion: 235±40.9 cells, n=3 animals; 6 weeks post-lesion: 348±67.3 cells; n=4 animals). This suggests that newly generated motor neurons matured and replaced lost motor neurons.

To directly demonstrate the presence of newly generated, terminally differentiated motor neurons, we used triple-labeling of BrdU (injected at 0, 2, and 4 days post.-lesion), ChAT and SV2 at 6 weeks post-lesion. In the 1500 μm surrounding the lesion site, we found 29±23.1 large BrdU⁺/ChAT⁺/cells (n=3 animals) that were covered with SV2⁺ contacts at a density that was comparable to that of motor neurons in unlesioned animals (FIG. 4C). Application of the axonal tracer biocytin to the muscle periphery labeled one BrdU+ cell in a motor neuron position in the ventro-medial spinal cord near the lesion site (n=8 animals, analyzed between 6 and 14 weeks post-lesion; FIG. 4D). This suggests that some newly generated motor neurons were integrated into the spinal circuitry and grew an axon out of the spinal cord.

### Discussion

We show here for the first time that a spinal lesion triggers generation of motor neurons in the spinal cord of adult zebrafish. Lesion-induced proliferation and motor neuron marker expression in olig2⁺ ependymo-radial glial cells makes these the likely motor neuron progenitor cells. Some of the newly generated motor neurons show markers for terminal differentiation and network integration.

Newly-generated motor neurons are added to pre-existing spinal tissue adjacent to a spinal lesion site in which normal cytoarchitecture is not restored. Thus, this model differs significantly from tail regeneration paradigms in amphibians in which the entire spinal cord tissue is completely reconstructed from an advancing blastema (Echeverri and Tanaka, 2002).

Our results clearly suggest olig2⁺ ependymo-radial glial cells to be the progenitor cells for spinal motor neurons, as a lesion induces their proliferation, and lineage tracing in olig2:GFP transgenic fish indicates that a substantial number of these cells acquire HB9 and/or islet-1/-2 expression and a third of olig2:GFP+/HB9+ cells were additionally labeled with BrdU after a lesion. Moreover, parenchymal olig2:GFP+ cells were never, and ependymo-radial glial cells outside the olig2:GFP+ zone were rarely labelled by HB9 or islet-1/-2 antibodies. This supports the notion that olig2:GFP+ ependymo-radial glial cells are the main source of motor neurons after a lesion. However, we cannot exclude the possibility that some motor neurons might have regenerated from olig2-negative parenchymal progenitors.

During post-embryonic development, olig2:GFP⁺ cells only give rise to oligodendrocytes (Park et al., 2007). Thus, adult neuronal regeneration is not just a continuation of a late developmental process, but an indication of significant plasticity of adult spinal progenitor cells in the fully mature spinal cord.

Additionally, olig2⁺ ependymo-radial glial cells have characteristics of neural stem cells. Our label-retention experiments indicate that some olig2:GFP⁺ ventricular cells are slowly-proliferating after a lesion, which is a stem cell characteristic (Pinto and Gotz, 2007). Lesion induced proliferation of these cells leads only to a moderate increase in their number, suggesting asymmetric cell divisions and some potential for self-renewal. Moreover, these cells express BLBP, which is also expressed in mammalian radial glial stem cells, and the PAR complex protein aPKC, an indicator of asymmetric cell division, at post-embryonic stages (Park et al., 2007). A stem cell role for olig2⁺ ependymo-radial glial cells would be in agreement with that of other radial glia cell types in developing mammals and in adult zebrafish (Pinto and Gotz, 2007). For example, Müller cells, the radial glia cell type in the adult retina, can produce different cell types in adult zebrafish, depending on which cells are lost after specific lesions (Fausett and Goldman, 2006; Bernardos et al., 2007; Fimbel et al., 2007).

After spinal cord lesion, we observed that numbers of differentiated motor neurons, i.e. large HB9:GFP⁺ cells and ChAT⁺ cells, were reduced at 2 weeks post-lesion and recovered at 6 to 8 weeks post-lesion. This suggests that motor neurons regenerate and is in agreement with previous observations in the guppy (Poecilia reticulata), in which large “ganglion cells” disappeared and reappeared after a lesion (Kirsche, 1950). In accordance with this finding, we detected terminally differentiated (ChAT⁺), newly-generated (BrdU⁺) motor neurons that were covered by SV⁺ contacts at 6 to 8 weeks post-lesion, suggesting their integration into the spinal network. The rare observation of one BrdU+ cell that is traced from the muscle periphery indicates that newly generated motor neurons ma even be capable of growing their axons out of the spinal cord towards muscle targets. In contrast, at early time points a transient population of small, newly-generated motor neurons (HB9:GFP⁺) that were not fully differentiated (ChAT⁻) and not decorated by SV2⁺ contacts were present in large numbers. These cells varied in motor neuron marker expression and the extent of process elaboration. Together, these observations suggest that motor neurons are generated and undergo successive steps of differentiation in terms of morphology and gene expression towards integration into an existing spinal network after a lesion.

In the lesioned spinal cord of mammals, proliferation and expression of nestin, an intermediate filament marker for progenitor cells, is increased around the ventricle and in parenchymal astrocytes, some of which carry radial processes (Yamamoto et al., 2001; Shibuya et al., 2002). Expression of Pax6, a transcription factor of progenitor cells in the embryonic spinal cord, is increased in the ependymal layer of the lesioned adult mammalian spinal cord; however, olig2 and several other factors are not re-expressed (Yamamoto et al., 2001; Ohori et al., 2006). Nevertheless, these observations suggest that spinal progenitors that exist in adult mammals (Shihabuddin et al., 2000) show some plasticity after a lesion and could potentially be induced to produce new motor neurons.

We conclude that the zebrafish, a powerful genetically tractable model, provides an opportunity to identify the evolutionarily conserved signals that trigger massive stem cell derived regeneration and network integration of motor neurons in the adult spinal cord.

### REFERENCES

Adolf B, Chapouton P, Lam C S, Topp S, Tannhauser B, Strähle U, Götz M, Bally-Cuif L (2006) Conserved and acquired features of adult neurogenesis in the zebrafish telencephalon. Dev Biol 295:278-293.
Arvidsson U, Riedl M, Elde R, Meister B (1997) Vesicular acetylcholine transporter (VAChT) protein: a novel and unique marker for cholinergic neurons in the central and peripheral nervous systems. J Comp Neurol 378:454-467.
Bareyre F M (2007) Neuronal repair and replacement in spinal cord injury. J Neurol Sci.
Becker C G, Lieberoth B C, Morellini F, Feldner J, Becker T, Schachner M (2004) L1.1 is involved in spinal cord regeneration in adult zebrafish. J Neurosci 24:7837-7842.
Becker T, Becker C G (2001) Regenerating descending axons preferentially reroute to the gray matter in the presence of a general macrophage/microglial reaction caudal to a spinal transection in adult zebrafish. J Comp Neurol 433:131-147.
Becker T, Lieberoth B C, Becker C G, Schachner M (2005) Differences in the regenerative response of neuronal cell populations and indications for plasticity in intraspinal neurons after spinal cord transection in adult zebrafish. Mol Cell Neurosci 30:265-278.
Becker T, Wullimann M F, Becker C G, Bernhardt R R, Schachner M (1997) Axonal regrowth after spinal cord transection in adult zebrafish. J Comp Neurol 377:577-595.
Bemardos R L, Barthel L K, Meyers J R, Raymond P A (2007) Late-stage neuronal progenitors in the retina are radial Muller glia that function as retinal stem cells. J Neurosci 27:7028-7040.
Chapouton P, Adolf B, Leucht C, Tannhauser B, Ryu S, Driever W, Bally-Cuif L (2006) her5 expression reveals a pool of neural stem cells in the adult zebrafish midbrain. Development 133:4293-4303.
Cheesman S E, Layden M J, Von Ohien T, Doe C Q, Eisen J S (2004) Zebrafish and fly Nkx6 proteins have similar CNS expression patterns and regulate motoneuron formation. Development 131:5221-5232.
Echeverri K, Tanaka E M (2002) Ectoderm to mesoderm lineage switching during axolotl tail regeneration. Science 298:1993-1996.
Fausett B V, Goldman D (2006) A role for alpha1 tubulin-expressing Muller glia in regeneration of the injured zebrafish retina. J Neurosci 26:6303-6313.
Fimbel S M, Montgomery J E, Burket C T, Hyde D R (2007) Regeneration of inner retinal neurons after intravitreal injection of ouabain in zebrafish. J Neurosci 27:1712-1724.
Flanagan-Steet H, Fox M A, Meyer D, Sanes J R (2005) Neuromuscular synapses can form in vivo by incorporation of initially aneural postsynaptic specializations. Development 132:4471-4481.
Fuccillo M, Joyner A L, Fishell G (2006) Morphogen to mitogen: the multiple roles of hedgehog signalling in vertebrate neural development. Nat Rev Neurosci 7:772-783.
Gould E (2007) How widespread is adult neurogenesis in mammals? Nat Rev Neurosci 8:481-488.
Grandel H, Kaslin J, Ganz J, Wenzel I, Brand M (2006) Neural stem cells and neurogenesis in the adult zebrafish brain: origin, proliferation dynamics, migration and cell fate. Dev Biol 295:263-277.
Higashijima S, Hotta Y, Okamoto H (2000) Visualization of cranial motor neurons in live transgenic zebrafish expressing green fluorescent protein under the control of the islet-1 promoter/enhancer. J Neurosci 20:206-218.
Jessell T M (2000) Neuronal specification in the spinal cord: inductive signals and transcriptional codes. Nat Rev Genet. 1:20-29.
Kirsche W (1950) Die regenerativen Vorgänge am Rückenmark erwachsener Teleostier nach operativer Kontinuitätstrennung. Z Mikrosk Anat Forsch 77:313-406.
Ninkovic J, Gotz M (2007) Signaling in adult neurogenesis: from stem cell niche to neuronal networks. Curr Opin Neurobiol 17:338-344.
Ohori Y, Yamamoto S, Nagao M, Sugimori M, Yamamoto N, Nakamura K, Nakafuku M (2006) Growth factor treatment and genetic manipulation stimulate neurogenesis and oligodendrogenesis by endogenous neural progenitors in the injured adult spinal cord. J Neurosci 26:11948-11960.
Park H C, Shin J, Appel B (2004) Spatial and temporal regulation of ventral spinal cord precursor specification by Hedgehog signaling. Development 131:5959-5969.
Park H C, Shin J, Roberts R K, Appel B (2007) An olig2 reporter gene marks oligodendrocyte precursors in the postembryonic spinal cord of zebrafish. Dev Dyn 236:3402-3407.
Pasinelli P, Brown R H (2006) Molecular biology of amyotrophic lateral sclerosis: insights from genetics. Nat Rev Neurosci 7:710-723.
Pinto L, Gotz M (2007) Radial glial cell heterogeneity—The source of diverse progeny in the CNS. Prog Neurobiol 83:2-23.
Poss K D, Wilson L G, Keating M T (2002) Heart regeneration in zebrafish. Science 298:2188-2190.
Shibuya S, Miyamoto O, Auer R N, Itano T, Mori S, Norimatsu H (2002) Embryonic intermediate filament, nestin, expression following traumatic spinal cord injury in adult rats. Neuroscience 114:905-916.
Shihabuddin L S, Horner P J, Ray J, Gage F H (2000) Adult spinal cord stem cells generate neurons after transplantation in the adult dentate gyrus. J Neurosci 20:8727-8735.
Shin J, Park H C, Topczewska J M, Mawdsley D J, Appel B (2003) Neural cell fate analysis in zebrafish using olig2 BAC transgenics. Methods Cell Sci 25:7-14.
Tsuchida T, Ensini M, Morton S B, Baldassare M, Edlund T, Jessell T M, Pfaff S L (1994) Topographic organization of embryonic motor neurons defined by expression of LIM homeobox genes. Cell 79:957-970.
Westerfield M (1989) The zebrafish book: a guide for the laboratory use of zebrafish (Brachydanio rerio). Eugene: University of Oregon Press.
William C M, Tanabe Y, Jessell T M (2003) Regulation of motor neuron subtype identity by repressor activity of Mnx class homeodomain proteins. Development 130:1523-1536.
Yamamoto S, Nagao M, Sugimori M, Kosako H, Nakatomi H, Yamamoto N, Takebayashi H, Nabeshima Y, Kitamura T, Weinmaster G, Nakamura K, Nakafuku M (2001) Transcription factor expression and Notch-dependent regulation of neural progenitors in the adult rat spinal cord. J Neurosci 21:9814-9823.
Zupanc G K, Hinsch K, Gage F H (2005) Proliferation, migration, neuronal differentiation, and long-term survival of new cells in the adult zebrafish brain. Comp Neurol 488:290-319.
Zupanc G K H, Horschke I (1995) Proliferation zones in the brain of adult gymnotiform fish: a quantitative mapping study. J Comp Neurol 353:213-233.

### Pharmacological Manipulation of Motor Neuron Regeneration

Motor neuron degenerative diseases (MNDs), such as amyotrophic lateral sclerosis (ALS) are devastating, because lost motor neurons do not regenerate. We have established tools for screening whole organisms for motor neuron differentiation using transgenic embryonic zebrafish, as well as the only paradigm in which adult spinal motor neurons regenerate (Reimer et al., 2008).

In the UK, the incidence of ALS is 2 in 100.000 and patients diagnosed with ALS have a life expectancy of 2-3 years. Riluzole is the only drug available that slows down, but does not halt disease progression (McDermott and Shaw, 2008). There is hope that stem cell therapy could be used to replace lost motor neurons. Preferably, endogenous spinal stem cells would be enticed to fully differentiate into functional (motor) neurons. These stem cells exist and show a limited regenerative response in mouse models of ALS (Shihabuddin et al., 2000; Chi et al., 2007; Juan et al., 2007). Small molecules can easily be delivered by intraperitoneal injection and could be used to drive differentiation of these cells further along the route to motor neuron regeneration. To discover small molecules that control motor neuron regeneration, we screen for compounds that influence motor neuron differentiation in embryonic zebrafish and validate hits in our adult motor neuron regeneration paradigm. The predictability for mammalian systems of molecule functions found in zebrafish is generally very good, such that zebrafish are currently used in drug toxicity tests (Zon and Peterson, 2005). Screening in whole vertebrate embryos has the advantage that toxicity, organ-specificity and bio-availability is already taken into account. As our screen will include known pharmaceutically active compounds, some of our hits could be developed into drugs relatively quickly.

As a first step towards translating our findings to mammalian models establish the endogenous response of stem cells to motor neuron loss in a mouse models of ALS. This puts us into a position to test newly found small molecules in mammalian neurodegeneration. Compounds could eventually be used to differentiate endogenous as well as transplanted stem cells into motor neurons. Small molecules that control neural stem cell differentiation may also be useful in other conditions in which lost neurons are not replaced, such as Parkinson's disease, Alzheimer's disease or spinal cord injury.

Described herein is a screen for small molecules that influence differentiation of motor neurons in a three step process, a primary screen in HB9:GFP transgenic fish, a secondary screen in islet-1:GFP transgenic fish, and a validation step in our adult spinal cord regeneration model. In parallel, it is possible to quantitatively assess a series of immunohistochemical markers for stem cell differentiation in a mouse ALS model in order to determine effects of small molecules found in the zebrafish system on motor neuron differentiation.

### BACKGROUND

Embryonic zebrafish are the major vertebrate model in which whole-organism small molecule screens have been performed. Their small size and aquatic development makes it easy to apply compounds. Previous screens were mainly based on altered morphology of embryos (Zon and Peterson, 2005; Sachidanandan et al., 2008; Yu et al., 2008), however, with the availability of transgenic reporter lines in zebrafish and the complete transparency of the embryos it is possible to rapidly screen for specific organs or cell types in living embryos.

We use two transgenic lines, in which expression of green fluorescent protein (GFP) is controlled by the motor neuron specific promoters HB9 or islet-1 in the spinal cord. In the HB9:GFP line (Flanagan-Steet et al., 2005), primary motor neurons and their ventral axons are labelled at 24 hours post-fertilisation (hpf). Aberrations in the highly stereotypic pattern of primary motor axons are easily detectable in a stereo-microscope (FIG. 8). In the islet-1:GFP line (Uemura et al., 2005), early spinal motor neurons are not labelled, however at around 48 hpf a subset of dorsally projecting motor neurons appears that can be easily visualised as a continuous band of cells along the ventral edge of the spinal cord in a stereo-microscope (FIG. 9). Thus, acceleration or delay of motor neuron differentiation can readily be assessed under a stereo-microscope.

Motor neurons regenerate in the lesioned spinal cord of adult zebrafish (Reimer et al., 2008). We have found motor neuron differentiation during regeneration to closely resemble the developmental situation. However, it is important to validate screen results from embryonic zebrafish in an adult regeneration paradigm. This is because differentiation processes during regeneration may differ from development, as demonstrated for heart regeneration in zebrafish (Raya et al., 2003).

Adult zebrafish are capable of functional regeneration after complete transection of the spinal cord (Becker et al., 2004). We find that after a spinal lesion, the ventricular zone shows a wide-spread increase in proliferation, including slowly proliferating olig2⁺ ependymo-radial glial progenitor cells. Lineage tracing in olig2:GFP transgenic fish indicates that these cells switch from a gliogenic phenotype to motor neuron production. Numbers of undifferentiated small HB9⁺, and islet-1⁺ motor neurons, which are double-labelled with the proliferation marker BrdU, are transiently strongly increased in the lesioned spinal cord. Large differentiated motor neurons, which are lost after a lesion re-appear at six to eight weeks post-lesion and we detected ChAT⁺/BrdU⁺ motor neurons covered by contacts immuno-positive for the synaptic marker SV2 (FIG. 10A,B). These observations suggest that after a lesion, plasticity of olig2⁺ progenitor cells may allow them to generate different types of motor neurons, some of which exhibit markers for terminal differentiation and integration into the existing adult spinal circuitry. The number of motor neurons produced is quantifiable and preliminary experiments suggest that intraperitoneal injections of small molecules influence motor neuron regeneration (see below).

Mouse models of ALS show a limited regenerative response. Transgenic mice, over-expressing variants of human mutations in the superoxide dismutase 1 (SOD1) gene, show degeneration of spinal motor neurons in a dose dependent manner. For example, high copy numbers of the G93A mutation lead to paralysis and death of the animals by 5 to 6 month of age (Gurney et al., 1994), low copy numbers lead to death at around 8 to 9 months of age (Puttaparthi et al., 2002). Interestingly, during the cell death period, these mice show attempted regeneration as indicated by the increased expression of nestin, a neural progenitor marker (Liu and Martin, 2006; Chi et al., 2007; Juan et al., 2007). A few of these cells even double-label with the neuronal marker NeuN, suggesting neuronal differentiation (Juan et al., 2007). However, motor neuron differentiation has never been observed in the SOD1^{G93A} mice. These observations suggest the presence of spinal stem cells, which could be manipulated to give rise to motor neurons.

### Study Design:

Different small molecule libraries may be used for screening. It is possible to screen the Spectrum Collection of FDA approved drugs and natural products and other bioactive components (2000 compounds), the Diversity Set of the US National Cancer Institute (1990 compounds), the Tocriscreen library (1120 compounds) and the Prestwick Chemical Library (1120 compounds). Due to some overlap between libraries, we will test approximately 5600 individual compounds. All of these libraries are commercially available.
Primary screen: Compounds are applied to HB9:GFP embryos in 24 well plates at a concentration of 10-25 μM in accordance with other studies (Zon and Peterson, 2005) at 6 hpf (mid-gastrula) and trajectories of motor axons analysed at 24 hpf. Analysing 2-3 embryos per compound is sufficient, because the pattern of motor axon outgrowth is highly stereotypic, making this a robust and quick screening tool. No anaesthesia or other manipulations are necessary to observe primary motor axons. Missing, stunted, excessively branched or supernumerary motor axons (FIG. 8), which are easily detectable in a fluorescence-equipped stereo-microscope, are classified as hits. Apparently toxic substances will be re-evaluated at lower concentrations. Specificity of the effect will be established in dose-response experiments for each hit.
Secondary screen: To exclude non-specific effects on motor axons due to gross alterations of the embryos occurring during early drug application, and to more directly analyse incipient differentiation of motor neurons, we use the islet-1:GFP fish. Hit compounds from the primary screen are applied at 24 hpf, when early embryogenesis is complete, but before islet-1:GFP+ motor neurons have been born. The read-out of this screen is whether the rostro-caudal band of late born secondary motor neurons in the ventral spinal cord is complete. Living embryos are screened at two time points, shortly before (48 hpf) and after (72 hpf) differentiation of these neurons during unmanipulated embryogenesis. Retardation and acceleration of motor neuron differentiation can be assayed (potential ectopic, i.e. more dorsal differentiation of motor neuron would also be detectable). To do this, 20 embryos per treatment will be dechorionated, anesthetised (tricaine 1:10000) and analysed under a stereomicroscope. This number of embryos is necessary to reliably detect changes in the timing of motor neuron differentiation, making this test unsuitable as a primary screening tool. We will again use 10-25 μM per compound. Toxic compounds will be re-screened at lower concentrations.
Validation: To test whether hit compounds influence adult motor neuron regeneration they are applied to the adult motor neuron regeneration paradigm (Reimer et al., 2008). Compounds may be dissolved in DMSO or 45% (2-Hydroxypropyl)-beta-cyclodextrin (Sigma-Aldrich, UK), to improve solubility in water, and injected intraperitoneally. Injection concentrations will depend on active concentrations in embryos. According to our previous experience, injections of 0.2 mg/ml in a volume of 25 μl (equalling 10 mg/kg body weight) at 3, 6 and 9 days post-lesion are suitable. Numbers of small and large HB9:GFP+ motor neurons will be assessed at 14 days post-lesion, when motor neuron regeneration peaks (Reimer et al., 2008). Motor neuron numbers will be stereologically determined from confocal image stacks of representative 50 μm sections. Due to variability in regeneration (Becker et al., 1997), it may be necessary to analyse 10 animals per compound.
Positive control compounds have effects in all three steps of the screening process: To verify that this experimental setup is able to deliver functional small molecules we have tested a known antagonist (cyclopamine) and an agonist of the sonic hedgehog (shh) pathway, known to be important for embryonic motor neuron differentiation, in all three paradigms. In the primary screen paradigm, both compounds caused partial (Hh-agonist) or complete (cyclopamine) absence of motor axons in 24 hpf HB9:GFP embryos. Similarity of phenotypes could be due to blocked motor neuron differentiation (cyclopamine) and disruption of stem cell proliferation by premature differentiation (Hh-agonist). In both cases motor axons do not grow out. According to our screening criteria, both compounds would have been classified as hits.

In the secondary screen paradigm, cyclopamine retarded motor neuron differentiation (11% with a complete band of differentiated motor neurons vs. 82% in controls at 72 hpf, p<0.00001) and the Hh-agonist accelerated it (76% vs. 28% control embryos with a complete spinal band of differentiated motor neurons at 48 hpf, p<0.00001; FIG. 9).

In the adult validation paradigm, cyclopamine leads to a significant 50% reduction in the number of newly generated motor neurons. The number of newly generated motor neurons in HB9:GFP transgenic animals within 1.5 mm surrounding the lesion site was 377±45.7 (n=9 animals), compared to animals injected with the related, but ineffective substance tomatidine (747±42.2 cells; n=10 animals; p=0.0004; manuscript in preparation) at 2 weeks post-lesion. The agonist increases the number of differentiated, large differentiated motor neurons more than 3-fold (68±8.8, n=9 animal vs. 20±4.2 large motor neurons in tomatidine injected animals, n=10 animals; p=0.0008; manuscript in preparation; FIG. 10C) at 2 weeks post-lesion. Numbers of newly generated small motor neurons were unchanged by the agonist (not shown), suggesting that the agonist accelerated motor neuron differentiation, but did not influence proliferation. This demonstrates that compounds that are classified as hits in the primary and secondary screen paradigms for motor neuron development affect adult motor neuron regeneration in a predictable manner.

A small scale test screen already produced hits in the primary and secondary screening paradigm: We pre-selected 80 substances from the list of pharmaceutically active compounds library (LOPAC, Sigma), which strongly overlaps with the library of FDA approved drugs, for their ability to inhibit neurosphere proliferation (Diamandis et al., 2007). Of these compounds, 7 showed alterations of motor axons in our primary screen paradigm (FIG. 8) and only one substance was toxic. In the secondary screen paradigm, of six tested hits, three inhibited and, notably, two accelerated differentiation of motor neurons (p<0.005, n>18 embryos). One compound had no effect. This shows that our primary screen can rapidly identify hits that are confirmed to influence initial motor neuron differentiation in our secondary screen paradigm. We are currently testing these substances in our adult validation paradigm.
Expected outcome and time course of screen: Our hit rate in the pilot primary screen is 9% (7 of 80), because our array of compounds was pre-selected for activity in neural stem cells. We expect a hit rate of 1%, when entire libraries are tested, comparable to other studies (Zon and Peterson, 2005). Thus we expect to detect approximately 60 hits in the primary screen, of which at least 30 may be confirmed in the secondary screen. We can then test 10 compounds with the most pronounced effects in the adult validation paradigm. Results from positive control substances suggest that many of the embryonic hits will also affect regeneration (see above). We estimate that we can screen up to 250 compounds per week in the primary screen, such that all compounds can be screened in 8 to 9 months (allowing for re-screening of toxic substances and temporary shortage in eggs). We can screen 10 compounds per week in the secondary screening paradigm, such that results should be obtained within 2 to 3 months. In the validation process, due to the histological analysis necessary, we estimate that we can analyse 2 compounds per month, such that 10 compounds can be analysed in 5 months (total 17 months). In the unlikely event that none of the compounds show an effect on motor neuron regeneration, we will choose more compounds from the secondary screen and known small molecules that target relevant signalling pathways, such as the FGF or retinoic acid signalling pathways, for further analyses.

Immunohistochemical analysis of SOD1^{G93A} mice: We will use the low copy number strain of SOD1^{G93A} transgenic mice (strain established in Edinburgh), which develops motor deficits by 6 months of age and succumbs by 8 months of age, such that potential treatments can be extended over a wider range of time. To determine possible regenerative attempts in this transgenic mouse strain, we will establish an immuno-histological time course of different marker genes at 3 months (pre-symptomatic), 5.5 months (beginning of symptoms) and 7 months (fully blown disease) of age, compared to wild type litter mates. We will use antibodies to the proliferating nuclear cell antigen (PCNA) and/or phospho-histone antibodies, to determine whether disease progression leads to increased proliferation of cells in the ventricular zone or in the parenchyma. Nestin antibodies will be used to determine a possible increase in progenitor cells populations. Double-labelling with the NeuN antibody will show whether neurogenesis occurs. We will also use antibodies to motor neuron differentiation markers HB9 and islet-1/-2. To our knowledge, none of these markers have been used in the low copy number strain of SOD1^{G93A}, or in SOD1 transgenic mice at all (HB9, islet-1/-2). Subsequently, we will use double labelling with BrdU to directly demonstrate whether different cell types were newly generated.

HB9 is a marker for very early motor neuron differentiation, whereas islet-1/-2 is expressed by more differentiated motor neurons (William et al., 2003). Therefore, it is possible that an attempted regeneration will lead to expression of HB9 in some cells, whereas expression of islets may be less likely. Cell numbers will be stereologically determined in 50 μm sections, such that a baseline is obtained for future studies with small molecule injections. All of the antibodies are available to us.

### REFERENCES

Becker C G, Becker T (2008) Adult zebrafish as a model for succesful central nervous system regeneration. Rest Neurol Neurosci in press.
Becker C G, Lieberoth B C, Morellini F, Feldner J, Becker T, Schachner M (2004) L1.1 is involved in spinal cord regeneration in adult zebrafish. J Neurosci 24:7837-7842.
Becker T, McLane M A, Becker C G (2003) Integrin interactions contribute to pathfinding of ventral motor nerves in embryonic zebrafish. Mol Cell Neurosci 23:54-68.
Becker T, Lieberoth B C, Becker C G, Schachner M (2005) Differences in the regenerative response of neuronal cell populations and indications for plasticity in intraspinal neurons after spinal cord transection in adult zebrafish. Mol Cell Neurosci 30:265-278.
Becker T, Wullimann M F, Becker C G, Bernhardt R R, Schachner M (1997) Axonal regrowth after spinal cord transection in adult zebrafish. J Comp Neurol 377:577-595.
Becker T, Bernhardt R R, Reinhard E, Wullimann M F, Tongiorgi E, Schachner M (1998) Readiness of zebrafish brain neurons to regenerate a spinal axon correlates with differential expression of specific cell recognition molecules. J Neurosci 18:5789-5803.
Becker T, Ostendorff H P, Bossenz M, Schliiter A, Becker C G, Peirano R I, Bach I (2002) Multiple functions of LIM domain-binding CLIM/NLI/Ldb cofactors during zebrafish development. Mech Dev 117:73-83.
Bovenkamp D E, Goishi K, Bahary N, Davidson A J, Zhou Y, Becker T, Becker C G, Zon L I, Klagsbrun M (2004) Expression and mapping of duplicate neuropilin-1 and neuropilin-2 genes in developing zebrafish. Gene Expr Patterns 4:361-370.
Chi L, Gan L, Luo C, Lien L, Liu R (2007) Temporal response of neural progenitor cells to disease onset and progression in amyotrophic lateral sclerosis-like transgenic mice. Stem Cells Dev 16:579-588.
Diamandis P, Wildenhain J, Clarke I D, Sacher A G, Graham J, Bellows D S, Ling E K, Ward R J, Jamieson L G, Tyers M, Dirks P B (2007) Chemical genetics reveals a complex functional ground state of neural stem cells. Nat Chem Biol 3:268-273.
Feldner J, Reimer M M, Schweitzer J, Wendik B, Meyer D, Becker T, Becker C G (2007) PlexinA3 restricts spinal exit points and branching of trunk motor nerves in embryonic zebrafish. J Neurosci 27:4978-4983.
Feldner J, Becker T, Goishi K, Schweitzer J, Lee P, Schachner M, Klagsbrun M, Becker C G (2005) Neuropilin-1a is involved in trunk motor axon outgrowth in embryonic zebrafish. Dev Dyn 234:535-549.
Flanagan-Steet H, Fox M A, Meyer D, Sanes J R (2005) Neuromuscular synapses can form in vivo by incorporation of initially aneural postsynaptic specializations. Development 132:4471-4481.
Gimnopoulos D, Becker C G, Ostendorff H P, Bach I, Schachner M, Becker T (2002) Expression of the zebrafish recognition molecule F3/F11/contactin in a subset of differentiating neurons is regulated by cofactors associated with LIM domains. Mech Dev 119:S135-S141.
Gurney M E, Pu H, Chiu A Y, Dal Canto M C, Polchow C Y, Alexander D D, Caliendo J, Hentati A, Kwon Y W, Deng H X, et al. (1994) Motor neuron degeneration in mice that express a human Cu,Zn superoxide dismutase mutation. Science 264:1772-1775.
Juan L, Dawei Z, Julie A D (2007) Increased number and differentiation of neural precursor cells in the brainstem of superoxide dismutase 1(G93A) (G1H) transgenic mouse model of amyotrophic lateral sclerosis. Neurol Res 29:204-209.
Liu Z, Martin L J (2006) The adult neural stem and progenitor cell niche is altered in amyotrophic lateral sclerosis mouse brain. J Comp Neurol 497:468-488.
McDermott C J, Shaw P J (2008) Diagnosis and management of motor neurone disease. Bmj 336:658-662.
Puttaparthi K, Gitomer W L, Krishnan U, Son M, Rajendran B, Elliott J L (2002) Disease progression in a transgenic model of familial amyotrophic lateral sclerosis is dependent on both neuronal and non-neuronal zinc binding proteins. J Neurosci 22:8790-8796.
Raya A, Koth C M, Buscher D, Kawakami Y, Itoh T, Raya R M, Sternik G, Tsai H J, Rodriguez-Esteban C, Izpisua-Belmonte J C (2003) Activation of Notch signaling pathway precedes heart regeneration in zebrafish. Proceedings of the National Academy of Sciences of the United States of America 100 Suppl 1:11889-11895.
Reimer M M, Sörensen I, Kuscha V, Frank R E, Liu C, Becker C G, Becker T (2008) Motor neuron regeneration in adult zebrafish. J Neurosci in revision.
Sachidanandan C, Yeh J R, Peterson Q P, Peterson R T (2008) Identification of a novel retinoid by small molecule screening with zebrafish embryos. PLoS ONE 3:e1947.
Schweitzer J, Becker T, Lefebvre J, Granato M, Schachner M, Becker C G (2005) Tenascin-C is involved in motor axon outgrowth in the trunk of developing zebrafish. Dev Dyn 234:550-566.
Shihabuddin L S, Horner P J, Ray J, Gage F H (2000) Adult spinal cord stem cells generate neurons after transplantation in the adult dentate gyrus. J Neurosci 20:8727-8735.
Uemura O, Okada Y, Ando H, Guedj M, Higashijima S, Shimazaki T, Chino N, Okano H, Okamoto H (2005) Comparative functional genomics revealed conservation and diversification of three enhancers of the isl1 gene for motor and sensory neuron-specific expression. Dev Biol 278:587-606.
William C M, Tanabe Y, Jessell T M (2003) Regulation of motor neuron subtype identity by repressor activity of Mnx class homeodomain proteins. Development 130:1523-1536.
Yu P B, Hong C C, Sachidanandan C, Babitt J L, Deng D Y, Hoyng S A, Lin H Y, Bloch K D, Peterson R T (2008) Dorsomorphin inhibits BMP signals required for embryogenesis and iron metabolism. Nat Chem Biol 4:33-41.
Zon L I, Peterson R T (2005) In vivo drug discovery in the zebrafish. Nat Rev Drug Discov 4:35-44.

1. A method of identifying an agent potentially useful in the treatment of motor neuron degenerative diseases (MNDDs), said method comprising the step of contacting an agent with a zebrafish having a spinal lesion and determining the effect of said test agent on the growth, differentiation, development and/or regeneration of motor neurons, wherein agents potentially useful in the treatment of MNDDs, modulate the growth, differentiation, development and/or regeneration of motor neurons.

2. Use of a zebrafish to identify compounds potentially useful in the treatment of motor neuron degenerative diseases (MNDD).

3. The use of claim 2, wherein the zebrafish is modified to include a spinal cord lesion.

4. The method of claim 1, or method of claim 2, wherein the spinal lesion comprises a lesion between the vertebrae.

5. The method or use of any preceding claim, wherein the zebrafish comprises labelled or detectable motor axons/neurons.

6. The method of claim 1, 4 or 5, wherein the agent is added at 3, 6 and/or 9 days post lesion formation.

7. The method of claim 1, 4, 5 or 6, wherein the effect of the agent on the growth, differentiation, development and/or regeneration of motor neurons is assessed at between 11 and 19 days post lesion.

8. The method of claims 1 and 4-7, comprising the additional step of first contacting one or more agents with embryonic zebrafish and determining the effects of these agents on motor axon development, growth and/or proliferation.

9. The method of claim 8, wherein agents found to modulate motor axon development, growth and/or proliferation in embryonic zebrafish are subjected to the methods of claims 1, 4-7.

10. The method of claim 8 or 9, wherein the embryonic zebrafish are modified to comprise labelled or detectable motor axons/neurons.

11. The method of claims 8-10, wherein the agents are contacted with the embryonic zebrafish at approximately 3-9 hours post fertilisation.

12. The method of claims 8-11, wherein the effects of the agents on motor axon development, growth and/or proliferation are determined at approximately 21-27 hours post fertilisation.

13. The method of claims 1 and 4-12 comprising the additional step of screening agents in later-stage embryonic zebrafish at 21-27 hours post fertilisation.

14. The method of claim 13, wherein the additional step occurs after the additional first step of claim 8 and before the methods provided by claims 1 and 4-7.

15. The method of claims 13 and 14, wherein the agents contacted with later-stage embryonic zebrafish are those found by the additional step of claim 8, to modulate motor axon development, growth and/or proliferation.

16. The method of claims 13-15, wherein the agents are added to the later stage embryonic zebrafish at approximately 21-27 hours post fertilisation and the effects of the agents are determined at approximately 45-51 hours post fertilisation.

17. A method of identifying agents potentially useful in the treatment of MNDDs, said method comprising the steps of:
(a) contacting a test agent with an embryonic zebrafish and determining the modulatory effect of said test agent on the growth, development, differentiation and/or regeneration of one or more motor axons/neurons;
(b) identifying agents capable of modulating the growth, development and/or regeneration of the motor neurons/axons in the embryonic zebrafish of step (a);
(c) contacting said identified agents with later-stage embryonic zebrafish and determining the modulatory effect of these agents on the growth, development, differentiation and/or regeneration of the motor neurons/axons;
(d) identifying agents capable of modulating the growth, development, differentiation and/or regeneration of the motor neurons/axons in the embryonic zebrafish of step (c); and
(e) contacting agents identified in step (d) with an adult zebrafish having a spinal lesion and determining the modulatory effect of these agents on the growth, development, differentiation and/or regeneration of the motor neurons/axons;
wherein test agents identified in step (e) as being capable of modulating the growth, development, differentiation and/or regeneration of the motor neurons/axons are potentially useful in the treatment of MNDD.

18. Compounds identified by the methods of claims 1 and 4-17 for treating MNDD.

19. Use of a compound identified by the methods of claims 1 and 4-17 in the manufacture of a medicament for treating MNDD.

20. A pharmaceutical composition comprising one or more compounds identified by the methods of claims 1 and 4-17 for use in treating MNDD in association with a pharmaceutically acceptable excipient carrier or diluent.

21. Dopamine agonists for treating MNDDs.

22. A compound selected from the group consisting of:
(i) norapomorphine
(ii) apomorphine
(iii) cycloheximide
(iv) taxol
(v) mavastatin
(vi) 13-cis retinoic acid
(vii) methotrexate
for treating MNDD.

23. A compound having the formula: 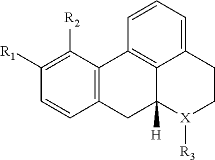
or a physiologically acceptable salt, solvate, ester or amide thereof,
wherein
X represents oxygen sulphur, or NH, or N when R₃ is present;
R₁ and R₂ are independently selected from the group consisting of hydrogen, halogen, nitro, cyano amide, hydroxyl, substituted or unsubstituted alkyl, substituted or unsubstituted alkenyl, substituted or unsubstituted aryl or heteroaryl, substituted or unsubstituted aralkyl, alkoxy, amino, mono- or di-alkyl substituted amino, sulphydryl, formyl, carboxyl, carboxylic acid, sulphonate, sulphonic acid, quaternary ammonium, C(═O)OR₄, C(═S)OR₄, C(═O)SR₄, C(═S)SR₄, C(═O)NH₂ and C(═S)NH₂ wherein one or both hydrogen atoms may be independently exchanged for R₄; and
R₃ and R₄ when present are each independently selected from the group consisting of substituted or unsubstituted alkyl, substituted or unsubstituted alkenyl, substituted or unsubstituted alkynyl, and substituted or unsubstituted —(CH₂)ₙ-aryl, wherein n is a number from 0 to 10; for treating MNDDs.
